(19) United States
(12) Patent Application Publication
(10) Pub. No.: US 2010/0150862 A1
(43) Pub. Date: Jun. 17, 2010

(54) METHODS OF MODULATING MATERNAL-FETAL TOLERANCE

(75) Inventor: Odile Devergne, Paris (FR)

Correspondence Address: MERCK;C/O SCHERING-PLOUGH BIOPHARMA, LEGAL DEPARTMENT, 901 CALIFORNIA AVENUE, PALO ALTO, CA, 94304, US

(73) Assignee: Schering Corporation

(21) Appl. No.: 12/516,458

(22) PCT Filed: Dec. 5, 2007
(86) PCT No.: PCT/US2007/024869

§ 371 Date: Jan. 14, 2010

Related U.S. Application Data
(60) Provisional application No. 60/869,035, filed on Dec. 7, 2006.

(51) Int. Cl. A61K 38/20; A61K 39/395; G01N 33/567; G01N 33/53; C12Q 1/68; A61P 37/02

(52) U.S. Cl. 424/85.2; 424/158.1; 435/7.21; 435/7.92; 435/6

(57) ABSTRACT

Provided are cytokines and methods of modulating maternal tolerance of a fetus using IL-27 agonists. Also provided are methods of modulating implantation of an embryo to a uterine lining, and methods of diagnosis.

### FIELD OF THE INVENTION

The present invention provides methods of modulating tolerance and implantation of an embryo on the uterine lining.

### BACKGROUND OF THE INVENTION

The mammalian immune response is based on a series of complex cellular interactions, called the “immune network”. Recent research has provided new insights into the inner workings of this network. While it remains clear that much of the response does, in fact, revolve around the network-like interactions of lymphocytes, macrophages, granulocytes, and other cells, immunologists now generally hold the opinion that soluble proteins, known as cytokines play a critical role in controlling these cellular interactions. Thus, there is considerable interest in the isolation, characterization, and mechanisms of action of cell modulatory factors, an understanding of which will lead to significant advancements in the diagnosis and therapy of numerous medical abnormalities, e.g., immune system disorders. Some of these factors are hematopoietic growth and/or differentiation factors, e.g., stem cell factor (SCF) or IL-12 (see, e.g., Mire-Sluis and Thorpe (1998) Cytokines, Academic Press, San Diego, Calif.; Thomson (ed.) (1998) The Cytokine Handbook (3d ed.) Academic Press, San Diego, Calif.; Metcalf and Nicola (1995) The Hematopoietic Colony Stimulating Factors, Cambridge Univ. Press, Cambridge, UK; and Aggarwal and Gutterman (1991) Human Cytokines, Blackwell, Malden, Mass.).

Cytokines mediate cellular activities in a variety of ways. They have been shown to support the proliferation, growth, and differentiation of pluripotential hematopoietic stem cells into large numbers of progenitors comprising diverse cellular lineages making up a complex immune system. Proper and balanced interactions between the cellular components are necessary for a healthy immune response. The different cellular lineages often respond in a different manner when cytokines are administered in conjunction with other agents.

Cell lineages especially important to the immune response include: B-cells, which can produce and secrete immunoglobulins (proteins with the capability of recognizing and binding to foreign matter to effect its removal), T-cells of various subsets that secrete cytokines and induce or suppress the B-cells and various other cells (including other T-cells) making up the immune network, NK cells, which are responsible for cytokine production in response to infectious agents and tumor cells, and antigen presenting cells such as dendritic and other myeloid derived cells.

The present invention provides methods of using IL-27, a cytokine related to IL-12. IL-12, a heterodimeric cytokine composed of two subunits, p35 and p40, plays a critical role in cell-mediated immunity. Its activities are triggered through a high-affinity receptor complex comprising two subunits, IL-12Rbeta1 and IL-12Rbeta2. The p35 subunit of IL-12 can bind to a second soluble protein called EBI3, and it was suggested that p35 and EBI3 form a secreted heterodimer, though the function of this heterodimer is unclear. EBI3 also binds to another protein, p28, to form a soluble heterodimer comprising p28 and EBI3, now called IL-27. The p28 subunit is also known as IL-80 or IL-D80. A cDNA encoding the human and mouse p35 subunit has been described in US20020164609 and WO 02/068596, both of which are incorporated by reference (see, e.g., Devergne, et al. (1997) Proc. Natl. Acad. Sci. USA 94:12041-12046; Chua, et al. (1995) J. Immunol. 155:4286:4294; Presky, et al. (1998) J. Immunol. 160:2174-2179; Gately, et al. (1998) Ann. Rev. Immunol. 16:495-521; Presky, et al. (1996) Proc. Natl. Acad. Sci. USA 93:14002-14007; Trinchieri (1998) Adv. Immunol. 70:83-243; Trinchieri (1998) Immunol. Res. 17:269-278; Trinchieri (1995) Annu. Rev. Immunol. 13:251-276).

In vitro studies have identified activated monocytes and monocyte-derived dendritic cells as the major source of IL-27. These studies also showed that the expression of EBI3 and p28 genes was not always coordinated. p28 gene was generally expressed at a lower level than EBI3 gene, very transiently, and by a more restricted spectrum of cell types (see, e.g., Pflanz et al. (2002) Immunity 16:779-790). Possibly due to this low and transient gene expression, in situ expression of p28 protein has been difficult to evidence. Immunohistochemical studies of EBI3 and p28 expression in human lymphoid tissues showed that EBI3 expression was in many cases not associated with detectable expression of p28, the co-expression of EBI3 and p28 being primarily detected in cells of the macrophage lineage and in endothelial cells of reactive tissues (see, e.g., Larousserie et al. (2004) J Pathol. 202:164-171; Larousserie et al. (2005) Am J Pathol 166:1217-1228; and Larousserie (2006) J Pathol 209:360-368).

Prior to the demonstration that EBI3 associates with p28 to form IL-27, it had been shown that EBI3 is expressed at a very high level at the human fetal-maternal interface (see, e.g., Devergne et al. (1996) J Virol 70:1143-1153; Devergne et al. (1997) Proc Natl Acad Sci USA 94:12041-12046; and Devergne et al. (2001) Am J Pathol 159:1763-1776). EBI3 is expressed throughout pregnancy by the only two cell types of fetal origin in direct contact with the maternal immune system: syncytiotrophoblasts, that interact with the maternal blood, and extravillous trophoblasts, that interact with the maternal uterine mucosa. Invasive extravillous trophoblasts play a key role in maternal tolerance and in vascular remodelling, in part through cytokine and chemokine production (see, e.g., Moffett and Loke (2006) Nat Rev Immunol 6:584-594). These cells specifically express HLA-G, a non-classical MHC class I molecule. By binding to different surface molecules, HLA-G displays immunoregulatory functions on several cell types including decidual NK cells, CD4⁺ and CD8⁺ T cells (see, e.g., Le Bouteiller et al. (2003)Placenta 24:Suppl A:S10-5.), and regulates angiogenesis (see, e.g., Fons et al. (2006) Blood 108:2608-2615.). Interestingly, purification of peptides presented by membrane-bound or soluble HLA-G revealed that a nonamer peptide from EBI3 was the most abundant peptide, accounting for up to 15% of all recovered ligand (see, e.g., Ishitani (2003) J Immunol 171:1376-1384).

From the foregoing, it is evident that discoveries of new functions and methods relating to cytokines and cytokine receptors, e.g., relating to IL-27 and its receptor complex, can contribute to new therapies for gestational abnormalities. In particular, the discovery and development of cytokines which enhance or potentiate the beneficial activities of known cytokines would be highly advantageous. The present invention provides methods of modulating tolerance and implantation of an embryo using IL-27.

### BRIEF DESCRIPTION OF THE DRAWINGS

FIG. 1 shows gene expression of EBI3, p28, IL-27R, and gp 130 from seven placentae collected at various terms of gestation, as analyzed by semi-quantitative RT-PCR. Magnetically purified human monocytes before stimulation or after overnight stimulation with LPS (100 ng/ml), were used as a positive control. The lane denoted <<->> corresponds to the negative control where no cDNA was added in the PCR reaction. β2-microglobulin (β2m) was used as an internal control for equal material in each condition.

FIG. 2A depicts co-immunoprecipitation of EBI3 with p28. NP40 extracts from two placentae collected at 17 and 28 weeks of gestation were submitted to immunoprecipitation (IP) with either rabbit polyclonal anti-EBI3 Abs, rabbit polyclonal anti-p28 Abs, or rabbit control Abs (10 μg per IP). A fraction of the cell lysate obtained before IP (lanes 2 and 5) and immunoprecipitates (lanes 1, 3, 4, 6, 7) were analyzed by SDS-PAGE on a 12% gel and subjected to western blot analysis with mouse monoclonal anti-EBI3 Ab or rabbit polyclonal anti-p28 Abs. The positions of EBI3 and p28 are indicated. Numbers at the left show positions of standard molecular weight proteins (in thousands). The band above the p28 band on the p28 blot most likely corresponds to rabbit Ig chains. Similar co-immunoprecipitation data were obtained using lysates from placentae of 9-, 33-, 36-, and 40-weeks of pregnancy.

FIG. 2B shows detection of IL-27 in the culture supernatant from placenta explants. Culture supernatants from explants of 14 term placentae collected at various times of culture as indicated in hours at the bottom of the graph, were tested by IL-27 ELISA. The mean value (±SD) is represented.

FIG. 3 shows sections from a first-trimester placenta that were analyzed by immunohistochemistry using: control rabbit Abs (a), rabbit polyclonal anti-p28 Abs (b, c) and mouse monoclonal anti-EBI3 Ab (d) as indicated at the top of each figure. In (c), a higher magnification of the zone delimitated by the frame in (b) is shown. Note that while syncytiotrophoblast cells are strongly positive for both EBI3 and p28, cytotrophoblast cells remain negative. Sections from a case of testicular mixed germ cell tumor with a choriocarcinoma component were stained with rabbit control Abs (e), rabbit polyclonal anti-p28 Abs (f), or mouse mononoclonal anti-EBI3 Ab (g). A syncytiotrophoblastic cell positive for both EBI3 and p28 is shown. In (f), the inset shows that, while syncytiotrophoblastic cells are positive for p28, cells morphologically consistent with cytotrophoblastic cells (arrows) are negative. Objectives: X10 in (a) and (b); X20 in (c-g). cc: cytotrophoblast cell; sy: syncytiotrophoblast cell.

FIG. 4 shows serial sections from a first-trimester placenta analyzed by immunohistochemistry with control rabbit Abs (a), rabbit anti-p28 Ab (b), or mouse anti-EBI3 Ab (c). Interstitial trophoblasts cells are positive for both EBI3 and p28. Objectives: X 20 in a-c. sa: spiral artery; evt: extravillous trophoblast cells.

### SUMMARY OF THE INVENTION

The present invention is based the discovery of IL-27 expression and production at sites of maternal-fetal interface.

The present invention provides a method of inducing maternal tolerance to a fetal antigen comprising administering to a subject at risk of or suffering from an immune mediated abortion, an effective amount of an IL-27 agonist. In one embodiment, the subject is human and the IL-27 agonist is administered during: a) first trimester of pregnancy; b) second trimester of pregnancy; c) third trimester of pregnancy; or d) first, second and third trimesters of pregnancy. The IL-27 agonist can be an IL-27 protein, which may be human IL-27. In certain embodiments, the IL-27 agonist is an agonist antibody that binds at least one subunit of an IL-27 receptor (IL-27R) complex. The IL-27R complex is human IL-27R complex. The agonist antibody induces signaling of the IL-27R complex.

The present invention also provides a method of enhancing implantation of at least one embryo to a uterine lining comprising administering to a subject an effective amount of an IL-27 agonist. The IL-27 agonist can be administered during: a) first trimester of pregnancy; b) second trimester of pregnancy; c) third trimester of pregnancy; or d) first, second, and third trimesters of pregnancy. The IL-27 agonist is contemplated to be an IL-27 protein. In a further embodiment, the IL-27 protein is human IL-27. It is also envisioned that the IL-27 agonists is an agonist antibody that binds at least one subunit of an IL-27R complex. The IL-27 complex is a human IL-27 complex, and the agonist antibody induces signaling of the IL-27R complex.

Also provided is a diagnostic method for determining whether a subject is suffering from immune mediated abortion comprising detecting the presence or level of IL-27 or IL-27R in a biological sample obtained from the subject, and determining whether the subject is having a spontaneous abortion. The biological sample can selected from the group consisting of a tissue sample, a cell sample, or a serum sample. In a further embodiment, the tissue sample is selected from the group consisting of a chorionic villus sample and a placental sample.

The present invention encompasses a prognostic method for determining whether a subject is at risk for developing immune mediated abortion comprising detecting the presence or level of IL-27 or IL-27R mRNA or polypeptide in a biological sample obtained from the subject, or isolate of the sample, thereby determining whether the subject is at risk for developing spontaneous abortion. The biological sample is selected from the group consisting of a tissue sample, a cell sample, or a serum sample, including a chorionic villus sample and a placental sample.

Also contemplated, is a method of monitoring IL-27 agonist treatment of a subject at risk of or suffering from an immune mediated abortion comprising measuring the levels of a Th1 or Th2 cytokine mRNA or polypeptide in a biological sample from the subject or an isolate of the sample, thereby determining if the subject is at risk of or suffering from an immune mediated abortion. The Th1 cytokine is selected from the group consisting of TNF-α, IFN-γ, IL-2, IL-1, IL-6, IL-12, and RANTES. The Th2 cytokine is selected from the group consisting of CSF-1, GM-CSF, IL-10, IL-4, IL-11, TGF and IL-3.

### DETAILED DESCRIPTION OF THE PREFERRED EMBODIMENTS

All references cited herein are incorporated herein by reference to the same extent as if each individual publication or patent application was specifically and individually indicated to be incorporated by reference.

As used herein, including the appended claims, the singular forms of words such as “a,” “an,” and “the,” include their corresponding plural references unless the context clearly dictates otherwise.

### I. DEFINITIONS

A molecule possesses at least one “IL-27 biological activity” or “IL-27 agonist activity” if the molecule can be recognized by an antibody raised against a native IL-27 protein; or if the molecule possesses any stimulatory, inhibitory or binding activity of a native IL-27 protein. For example, the molecule may enhance an immune cell to produce IFNgamma or the molecule may bind to an IL-27 receptor. The molecule preferably binds to WSX-1/TCCR, and more preferably is capable of enhancing IFNgamma production.

“Administration” and “treatment,” as it applies to a human, veterinary, animal, experimental subject, cell, tissue, organ, or biological fluid, refers to contact of an exogenous pharmaceutical, therapeutic, diagnostic agent, or composition to the animal, human, subject, cell, tissue, organ, or biological fluid. “Administration” and “treatment” can refer, e.g., to therapeutic, pharmacokinetic, diagnostic, research, and experimental methods. Treatment of a cell encompasses contact of a reagent to the cell, as well as contact of a reagent to a fluid, where the fluid is in contact with the cell. Treatment of a cell includes situations where the reagent contacts a biological fluid in a human or animal, but where the reagent has not been demonstrated to contact the cell. Treatment further encompasses situations where an administered reagent or cell is modified by metabolism, degradation, or by conditions of storage.

The term “enhancing” the biological activity, function, health, or condition of an organism refers to the process of augmenting, fortifying, strengthening, or improving.

The term “operably linked” means that the components to which the term is applied are in a relationship that allows them to carry out their inherent functions under suitable conditions. For example, a control sequence “operably linked” to a protein coding sequence is ligated thereto so that expression of the protein coding sequence is achieved under conditions compatible with the transcriptional activity of the control sequences.

The term “control sequence” means that the subject polynucleotide sequence can effect expression and processing of coding sequences to which it is ligated. The nature of such control sequences may depend upon the host organism. In particular embodiments, control sequences for prokaryotes may include a promoter, ribosomal binding site, and transcription termination sequence. In other particular embodiments, control sequences for eukaryotes may include promoters comprising one or a plurality of recognition sites for transcription factors, transcription enhancer sequences, and transcription termination sequence. In certain embodiments, “control sequences” can include leader sequences and/or fusion partner sequences.

The term “polynucleotide” means single-stranded or double-stranded nucleic acid polymers of at least 10 bases in length. In certain embodiments, the nucleotides comprising the polynucleotide can be ribonucleotides or deoxyribonucleotides or a modified form of either type of nucleotide. Said modifications include base modifications such as bromouridine and inosine derivatives, ribose modifications such as 2′,3′-dideoxyribose, and internucleotide linkage modifications such as phosphorothioate, phosphorodithioate, phosphoroselenoate, phosphorodiselenoate, phosphoroanilothioate, phoshoraniladate and phosphoroamidate. The term includes single and double stranded forms of DNA.

The term “oligonucleotide” means a polynucleotide comprising a length of 200 bases or fewer. In preferred embodiments, oligonucleotides are 10 to 60 bases in length. In more preferred embodiments, oligonucleotides are 12, 13, 14, 15, 16, 17, 18, 19, or 20 to 40 bases in length. Oligonucleotides may be single stranded or double stranded, e.g., for use in the construction of a mutant gene. Oligonucleotides of the invention may be sense or antisense oligonucleotides.

The term “naturally occurring nucleotides” includes deoxyribonucleotides and ribonucleotides. The term “modified nucleotides” includes nucleotides with modified or substituted sugar groups or modified or substituted bases. The term “oligonucleotide linkages” includes linkages such as phosphorothioate, phosphorodithioate, phosphoroselenoate, phosphorodiselenoate, phosphoroanilothioate, phoshoraniladate, phosphoroamidate, and the like. See, e.g., LaPlanche et al. (1986), Nucl. Acids Res. 14:9081; Stec et al. (1984), J. Am. Chem. Soc. 106:6077; Stein et al. (1988), Nucl. Acids Res. 16:3209; Zon et al. (1991), Anti-Cancer Drug Design 6:539; Zon et al. (1991), Oligonucleotides and Analogues: A Practical Approach, pp. 87-108 (F. Eckstein, ed.), Oxford University Press, Oxford England; Stec et al., U.S. Pat. No. 5,151,510; Uhlmann and Peyman (1990), Chemical Reviews 90:543, the disclosures of which are hereby incorporated by reference for any purpose. An oligonucleotide of the invention can include a label, including a radiolabel, a fluorescent label, a hapten or an antigenic label, for detection assays.

The term “vector” means any molecule (e.g., nucleic acid, plasmid, or virus) used to transfer coding information to a host cell.

The term “expression vector” or “expression construct” refers to a vector that is suitable for transformation of a host cell and contains nucleic acid sequences that direct and/or control (in conjunction with the host cell) expression of one or more heterologous coding regions operatively linked thereto. An expression construct may include, but is not limited to, sequences that affect or control transcription, translation, and RNA splicing, if introns are present, of a coding region operably linked thereto.

The term “host cell” means a cell that has been transformed, or is capable of being transformed, with a nucleic acid sequence and thereby expresses a selected gene of interest. The term includes the progeny of the parent cell, whether or not the progeny is identical in morphology or in genetic make-up to the original parent cell, so long as the selected gene is present.

The term “transduction” means the transfer of genes from one bacterium to another, usually by phage. “Transduction” also refers to the acquisition and transfer of eukaryotic cellular sequences by retroviruses.

The term “transfection” means the uptake of foreign or exogenous DNA by a cell, and a cell has been “transfected” when the exogenous DNA has been introduced inside the cell membrane. A number of transfection techniques are well known in the art and are disclosed herein. See, e.g., Graham et al., 1973, Virology 52:456; Sambrook et al., 2001, Molecular Cloning: A Laboratory Manual, Id.; Davis et al., 1986, Basic Methods in Molecular Biology, Elsevier; and Chu et al., 1981, Gene 13:197. Such techniques can be used to introduce one or more exogenous DNA moieties into suitable host cells.

The term “transformation” refers to a change in a cell's genetic characteristics, and a cell has been transformed when it has been modified to contain new DNA. For example, a cell is transformed where it is genetically modified from its native state by transfection, transduction, or other techniques. Following transfection or transduction, the transforming DNA may recombine with that of the cell by physically integrating into a chromosome of the cell, or may be maintained transiently as an episomal element without being replicated, or may replicate independently as a plasmid. A cell is considered to have been “stably transformed” when the transforming DNA is replicated with the division of the cell.

Methods of using conservatively modified variants, derivatives, and muteins of polypeptides and nucleic acids of IL-27 are provided. “Conservatively modified variants” applies to both amino acid and nucleic acid sequences. With respect to particular nucleic acid sequences, conservatively modified variants refers to those nucleic acids which encode identical or essentially identical amino acid sequences or, where the nucleic acid does not encode an amino acid sequence, to essentially identical nucleic acid sequences. Because of the degeneracy of the genetic code, a large number of functionally identical nucleic acids may encode any given protein.

As to amino acid sequences, one of skill will recognize that an individual substitution to a nucleic acid, peptide, polypeptide, or protein sequence which substitutes an amino acid or a small percentage of amino acids in the encoded sequence for a conserved amino acid is a “conservatively modified variant.” Conservative substitution tables providing functionally similar amino acids are well known in the art. An example of a conservative substitution is the exchange of an amino acid in one of the following groups for another amino acid of the same group (U.S. Pat. No. 5,767,063 issued to Lee, et al.; Kyte and Doolittle (1982) J. Mol. Biol. 157: 105-132):

### (1) Hydrophobic: Norleucine, Ile, Val, Leu, Phe, Cys, or Met;

(2) Neutral hydrophilic: Cys, Ser, Thr;

### (3) Acidic: Asp, Glu;

### (4) Basic: Asn, Gln, His, Lys, Arg;

(5) Residues that influence chain orientation: Gly, Pro;

### (6) Aromatic: Trp, Tyr, Phe;

(7) Small amino acids: Gly, Ala, Ser.

The terms “polypeptide” or “protein” means molecules having the sequence of native proteins, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. The terms “polypeptide” and “protein” specifically encompass antibodies, or sequences that have deletions from, additions to, and/or substitutions of one or more amino acid of such antibody. The term “polypeptide fragment” refers to a polypeptide that has an amino-terminal deletion, a carboxyl-terminal deletion, and/or an internal deletion. In certain embodiments, fragments are at least 5 to about 500 amino acids long. It will be appreciated that in certain embodiments, fragments are at least 5, 6, 8, 10, 14, 20, 50, 70, 100, 110, 150, 200, 250, 300, 350, 400, or 450 amino acids long. Particularly useful polypeptide fragments include functional domains, including binding domains. In the case of an antibody, useful fragments include but are not limited to a CDR region, a variable domain of a heavy or light chain, a portion of an antibody chain or just its variable region including two CDRs, and the like.

The terms “naturally occurring” and “native” mean that the biological materials (molecules, sequences, protein complexes, cells, and the like) to which the terms are applied can be found in nature and are not manipulated by man. For example, a polypeptide or polynucleotide sequence that is present in an organism (including viruses) that can be isolated from a source in nature and that has not been intentionally modified by man is naturally occurring. Likewise, the terms “non-naturally occurring” or “non-native” refer to a material that is not found in nature or that has been structurally modified or synthesized by man.

Peptide analogs are commonly used in the pharmaceutical industry as non-peptide drugs with properties analogous to those of the template peptide. These types of non-peptide compounds are termed “peptide mimetics” or “peptidomimetics”. See Fauchere (1986), Adv. Drug Res. 15:29; Veber & Freidinger, 1985, TINS p. 392; and Evans et al. (1987), J. Med. Chem. 30:1229, which are incorporated herein by reference. Such compounds are often developed with the aid of computerized molecular modeling. Peptide mimetics that are structurally similar to therapeutically useful peptides may be used to produce a similar therapeutic or prophylactic effect. Generally, peptidomimetics are structurally similar to a paradigm peptide or polypeptide (i.e., a peptide or polypeptide that has a biochemical property or pharmacological activity), such as human antibody, but have one or more peptide linkages optionally replaced by a linkage selected from: —CH₂—NH—, —CH₂—S—, —CH₂—CH₂—, —CH═CH—(cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) may be used in certain embodiments to generate more stable peptides. In addition, constrained peptides comprising a consensus sequence or a substantially identical consensus sequence variation may be generated by methods known in the art (Rizo & Gierasch, 1992, Ann. Rev. Biochem. 61:387, incorporated herein by reference for any purpose); for example, by adding internal cysteine residues capable of forming intramolecular disulfide bridges which cyclize the peptide.

The term “half-life extender” refers to a molecule that prevents degradation and/or increases half-life, reduces toxicity, reduces immunogenicity, or increases biological activity of a therapeutic protein. Exemplary vehicles include an Fc domain (which is preferred) as well as a linear polymer (e.g., polyethylene glycol (PEG), polylysine, dextran, etc.); a branched-chain polymer (see, for example, U.S. Pat. No. 4,289,872 to Denkenwalter et al., issued Sep. 15, 1981; U.S. Pat. No. 5,229,490 to Tam, issued Jul. 20, 1993; WO 93/21259 by Frechet et al., published 28 Oct. 1993); a lipid; a cholesterol group (such as a steroid); a carbohydrate or oligosaccharide (e.g., dextran); any natural or synthetic protein, polypeptide or peptide that binds to a salvage receptor; albumin, including human serum albumin (HSA), leucine zipper domain, and other such proteins and protein fragments.

The term “native Fc” refers to molecule or sequence comprising the sequence of a non-antigen-binding fragment resulting from digestion of whole antibody, whether in monomeric or multimeric form. The original immunoglobulin source of the native Fc is preferably of human origin and may be any of the immunoglobulins, although IgG1 and IgG2 are preferred. Native Fc's are made up of monomeric polypeptides that may be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, IgE) or subclass (e.g., IgG1, IgG2, IgG3, IgA1, IgGA2). One example of a native Fc is a disulfide-bonded dimer resulting from papain digestion of an IgG (see Ellison et al. (1982), Nucleic Acids Res. 10: 4071-9). The term “native Fc” as used herein is generic to the monomeric, dimeric, and multimeric forms.

The term “selective binding agent” refers to a molecule which preferentially binds a protein of interest. A selective binding agent may include a protein, peptide, nucleic acid, carbohydrate, lipid, or small molecular weight compound. Examples of proteins that are selective binding agents of the inventive subject matter include soluble receptors (i.e., proteins having all or part of the extracellular domain of a naturally occurring membrane-bound protein but not the transmembrane domain or intracellular domain); antibodies and fragments thereof; variants, derivatives and fusion proteins of antibodies and soluble receptors; peptidomimetic compounds; and organo-mimetic compounds. In a preferred embodiment, a selective binding agent is an antibody, such as polyclonal antibodies, monoclonal antibodies (mAbs), chimeric antibodies, CDR-grafted antibodies, anti-idiotypic (anti-Id) antibodies to antibodies that can be labeled in soluble or bound form, as well as fragments, regions or derivatives thereof, provided by known techniques, including, but not limited to enzymatic cleavage, peptide synthesis or recombinant techniques.

The term “antigen” refers to a molecule or a portion of a molecule capable of being bound by a selective binding agent, such as an antibody, and additionally capable of being used in an animal to produce antibodies capable of binding to an epitope of that antigen. An antigen may have one or more epitopes.

The term “epitope” includes any determinant, preferably a polypeptide determinant, capable of specific binding to an immunoglobulin or T-cell receptor. In certain embodiments, epitope determinants include chemically active surface groupings of molecules such as amino acids, sugar side chains, phosphoryl, or sulfonyl, and, in certain embodiments, may have specific three-dimensional structural characteristics, and/or specific charge characteristics. An epitope is a region of an antigen that is bound by an antibody. In certain embodiments, an antibody is said to specifically bind an antigen when it preferentially recognizes its target antigen in a complex mixture of proteins and/or macromolecules. In preferred embodiments, an antibody is said to specifically bind an antigen when the dissociation constant is less than or equal to about 10 nM, more preferably when the dissociation constant is less than or equal to about 100 pM, and most preferably when the dissociation constant is less than or equal to about 10 pM. “Antibody” or “antibody peptide(s)” refer to an intact antibody, or a binding fragment thereof that competes with the intact antibody for specific binding and includes chimeric, humanized, fully human, and bispecific antibodies. In certain embodiments, binding fragments are produced by recombinant DNA techniques. In additional embodiments, binding fragments are produced by enzymatic or chemical cleavage of intact antibodies. Binding fragments include, but are not limited to, Fab, Fab′, F(ab′)₂, Fv, immunologically functional immunoglobulin fragments, heavy chain, light chain, and single-chain antibodies.

The term “heavy chain” includes a full-length heavy chain and fragments thereof having sufficient variable region sequence to confer binding specificity. A full-length heavy chain includes a variable region domain, V_{H}, and three constant region domains, C_{H}1, C_{H}2, and C_{H}3. The V_{H} domain is at the amino-terminus of the polypeptide, and the CH³ domain is at the carboxyl-terminus.

The term “light chain” includes a full-length light chain and fragments thereof having sufficient variable region sequence to confer binding specificity. A full-length light chain includes a variable region domain, V_{L}, and a constant region domain, C_{L}. Like the heavy chain, the variable region domain of the light chain is at the amino-terminus of the polypeptide.

A “Fab fragment” is comprised of one light chain and the C_{H}1 and variable regions of one heavy chain. The heavy chain of a Fab molecule cannot form a disulfide bond with another heavy chain molecule.

A “Fab' fragment” contains one light chain and one heavy chain that contains more of the constant region, between the C_{H}1 and C_{H}2 domains, such that an interchain disulfide bond can be formed between two heavy chains to form a F(ab′)₂ molecule.

A “F(ab′)₂ fragment” contains two light chains and two heavy chains containing a portion of the constant region between the C_{H}1 and C_{H}2 domains, such that an interchain disulfide bond is formed between two heavy chains.

The “Fv region” comprises the variable regions from both the heavy and light chains, but lacks the constant regions.

“Single-chain antibodies” are Fv molecules in which the heavy and light chain variable regions have been connected by a flexible linker to form a single polypeptide chain, which forms an antigen-binding region. Single chain antibodies are discussed in detail in International Patent Application Publication No. WO 88/01649 and U.S. Pat. Nos. 4,946,778 and 5,260,203, the disclosures of which are incorporated by reference for any purpose.

A “bivalent antibody” other than a “multispecific” or “multifunctional” antibody, in certain embodiments, is understood to comprise binding sites having identical antigenic specificity.

A “bispecific” or “bifunctional” antibody is a hybrid antibody having two different heavy/light chain pairs and two different binding sites. Bispecific antibodies may be produced by a variety of methods including, but not limited to, fusion of hybridomas or linking of Fab' fragments. See, e.g., Songsivilai & Lachmann (1990), Clin. Exp. Immunol. 79:315-321; Kostelny et al. (1992), J. Immunol. 148:1547-1553.

An “effective amount” is an amount of a therapeutic agent sufficient to achieve the intended purpose. For example, an effective amount of a composition to enhance maternal tolerance is an amount sufficient to prevent immune rejection of a fetus, embryo, or fertilized egg. Similarly an effective amount of a composition will promote implantation or attachment of a fertilized egg or embryo on the uterine lining. An effective amount for treating or ameliorating a disorder, disease, or medical condition is an amount sufficient to result in a reduction or complete removal of the symptoms of the disorder, disease, or medical condition. The effective amount of a given therapeutic agent will vary with factors such as the nature of the agent, the route of administration, the size and species of the animal to receive the therapeutic agent, and the purpose of the administration. The effective amount in each individual case may be determined empirically by a skilled artisan according to established methods in the art.

“Exposing” a cell to a substance refers to providing the substance to the cell directly or indirectly. The substance can be provided indirectly, for example, by providing a precursor of the substance, which is known to be converted to the substance. For instance, exposing a target cell to IL-27 can be achieved by providing to the target cell a composition comprising the IL-27 protein, or by introducing the gene(s) coding for IL-27 into the target cell. Alternatively, it can also be achieved by introducing the gene(s) coding for IL-27 into a second cell and mixing the target cell with the second cell.

“Expression” encompasses the biosynthesis of a nucleic acid, e.g., mRNA, or of a polypeptide, as well as changes in the compartmentalization of a macromolecule, e.g., by traversal from the nucleus to the cytosol, insertion into the plasma membrane, degranulation, or secretion. Expression or production of a macromolecule by the cell may include only the amount found in the cell, e.g., in a cell homogenate, at a given point in time. Generally, this definition applies to expression of non-secreted molecules. Alternatively, expression or production of a macromolecule by a cell includes the amount found in the cell plus the amount secreted and accumulated, e.g., in a cell medium or biological compartment. Generally, this definition applies to secreted or degranulated proteins, e.g., cytokines. “Levels” refers to concentrations in a compartment, including a biological compartment, e.g., in a predetermined volume of, e.g., plasma, serum, blood, interstitial fluid, cerebrospinal fluid, or urine, in a whole organ or fragment of the organ, in a compartment within an organ, e.g., red pulp, white pulp, or pancreatic islets, or in a specific cell or group of cells, e.g., macrophages.

A “hyperkine” is an engineered heterodimeric, homodimeric, or multimeric cytokine wherein at least two cytokine polypeptide subunits of the cytokine are covalently associated to each other (Pflanz, et al. (2002) Immunity 16:779-790). In the case of IL-27, a hyperkine is the covelent association of p28 and EBI3.

An “immune cell” is a cell of the immune system, such as B cell, T cell, NK cell, monocyte, macrophage, mast cell, eosinophil, or antigen presenting cell (APC), or dendritic cell. Depending on the context, an immune cell can also be any cell that expresses mediators of immunity, an epithelial cell that expresses cytokines, depending on the context.

An human or animal subject “suspected of having” a disorder, disease, or medical condition is one that is not yet diagnosed as having the disorder, disease, or medical condition, but that shows one or more symptoms of the disorder, has a genetic disposition for the disorder, or has been previously treated for disorder, where the disorder is subject to recurrence.

“Nucleic acid” encompasses single stranded nucleic acids as well as double stranded nucleic acids comprised of a complex of a single stranded nucleic acid strand and its complementary strand. The present invention encompasses methods of using at least one nucleic acid, e.g., residing in at least one expression vector, comprising at least one nucleic acid encoding each subunit of the IL-27 heterodimeric cytokine.

The invention contemplates methods where, e.g., the above nucleic acids are encoded by one vector; where one nucleic acid is encoded by a first vector and where the remaining nucleic acids are encoded by a second vector; and where each nucleic acid is encoded by separate, respective vectors, and various combinations thereof. Also contemplated are methods that provide the above cytokines, where one or more cytokines are provided by a vector and where one or more cytokines are directly provided by a cytokine polypeptide, e.g., treatment with a composition comprising a vector and a polypeptide. The vectors of the contemplated method comprise, e.g., a first promoter operably linked with a first nucleic acid; a second promoter operably linked with a second nucleic acid; a third promoter operably linked with a third nucleic acid, and the like, as well as a first promoter operably linked with a first and second nucleic acid, a first promoter operably linked with a first, second, and third nucleic acid, and the like.

“Treating or ameliorating” means the reduction or complete removal of the symptoms of a disorder, disease, or medical condition.

As used herein, the term “biological sample” refers to a sample of biological material isolated from a subject, preferably a human subject, or present within a subject, preferably a human subject. The “biological material” can include, for example, tissues, tissue samples, tumors, tumor samples, cells, biological fluids, and purified and/or partially-purified biological molecules. As used herein, the term “isolated”, when used in the context of a biological sample, is intended to indicate that the biological sample has been removed from the subject.). The level of IL-27 or IL-27R in maternal serum or blood is determined. In a further another embodiment, the level of IL-27 or IL-27R can be determined in a placental, amniotic fluid or chorionic villous sample.

As used herein the term “immune mediated abortion” includes spontaneous termination of a pregnancy, e.g., a miscarriage. An immune mediated abortion as defined herein includes failure of a fertilized embryo to properly implant in the wall of the uterus of a subject or, once implanted, shedding of the embryo from the uterus of the subject, or reabsorption of an embryo by the subject. An immune-mediated abortion arises from an immune response by a subject to an antigen, e.g., an embryonic or fetal antigen or paternal antigen expressed by or released from the embryo or fetus.

As used herein, the term “implantation” includes attachment of the fertilized egg to the uterine lining following a natural fertilization process or with the aid of assisted reproductive technology. As used herein “assisted reproductive technology” refers to any technically assisted method of reproduction such as, for example ovulation induction, in vitro fertilization, embryo transfer, and the like. In humans implantation usually occurs five to seven days after ovulation in the natural reproductive process.

As used herein the term “embryo” includes a stage in the development of a multicelled organism between the time that the zygote is fertilized and the point at which the organism developing from that zygote becomes free-living.

“Tolerance” encompasses immune tolerance which is an unresponsiveness to, e.g., a cancer or tumor, or to an alloantigen, such as a graft alloantigen, to a foreign antigenic molecule, to a foreign molecular complex, or to an antigen from a foreign organism or virus. Tolerance also encompasses immune unresponsiveness, or an increase in immune unresponsiveness, to an fetal antigen during a pregnancy. Additionally, “tolerance” encompasses naturally occurring tolerance and artificially or pharmacologically induced tolerance. Moreover, tolerance also relates to immune unresponsiveness to self antigens that are recognized by molecular mimicry (see, e.g., Liu (1997) J. Exp. Med. 186:625-629; Waldman and Cobbold (1998) Annul Rev. Immunol. 16:619-644; Xiao and Link(1997) Clin. Immunol. Immunopathol. 85:119-128; Steinman, et al. (2003) Annu. Rev. Immunol. 21:685-711; Olson, et al. (2002) J. Immunol. 169:2719-2726; Toussirot (2002) Curr. Drugs Targets Inflamm. Allergy 1:45-52; Takahashi and Sakaguchi (2003) Int Rev CytoL 225:1-32; Burt, et al. (2002) Int. J. Hematol. 76 (Suppl 1):226-47; Gery and Egwuagu (2002) Int. Rev. Immunol. 21(2-3):89-100; Weiner (2001) Microbes Infect. 3:947-54). In the present invention, “maternal tolerance” refers to tolerance of a fetal antigen or a paternal antigen expressed or released by the fetus or embryo.

### II. GENERAL

The present invention provides a method of modulating maternal tolerance of a fetus. Further provided is a method of modulating implantation of an embryo on a uterine lining.

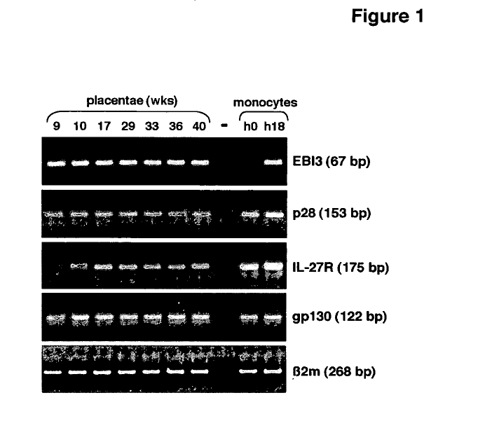

The expression of EBI3 and p28 genes in 7 normal human placenta ranging from 9 to 40 weeks of gestation was studied. Purified monocytes, either unstimulated or stimulated for 18 hours with LPS, were used as a positive control. In LPS-stimulated monocytes and in all cases of placenta, a signal for EBI3 and p28 was detected, indicating that the two subunits of IL-27 are expressed in the placenta at various gestational ages. Similarly, gene expression of the two components of the IL-27 receptor, IL-27R and gp130, was detected in all placenta (FIG. 1). Thus, genes encoding IL-27 and the IL-27R complex are expressed in the placenta at different stages of pregnancy.

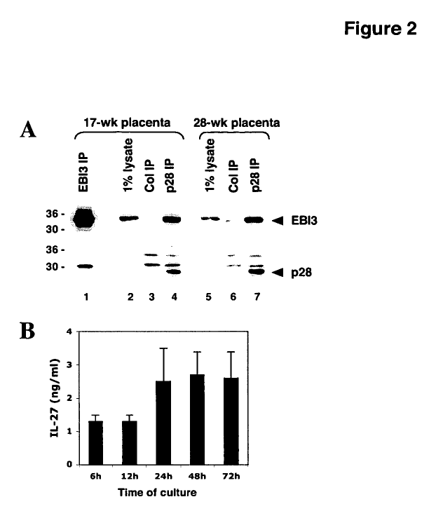

Next, the protein level IL-27 heterodimer in the human placenta was investigated. To this end, 0.5% NP40 extracts were prepared from placentae at various stages of pregnancy and submitted to co-immunoprecipitation experiments (FIG. 2A). p28 was immunoprecipitated with rabbit anti-p28 Abs or control rabbit IgG, and the amount of associated EBI3 was evaluated by EBI3 western blot. As a positive control, lysates were submitted to immunoprecipitation with rabbit anti-EBI3 Abs. As expected, EBI3 immunoblot identified a 33-kDa protein in the placental lysates before immunoprecipitation (FIG. 2A, top panel, lanes 2 and 5), and in the anti-EBI3 immunoprecipitate (FIG. 2A, top panel, lane 1). More importantly, EBI3 was also detected in anti-p28 immunoprecipitates (FIG. 2A, top panel, lanes 4 and 7), but was barely detectable in control immunoprecipitates (lanes 3 and 6), indicating that EBI3 was specifically associated with p28. p28 was not detectable by western blot in the placental lysates, unless it was immunoprecipitated with anti-p28 Abs (FIG. 2, bottom panel). Also, no p28 band was observed by western blot in the anti-EBI3 immunoprecipitate (FIG. 2A, bottom panel, lane 1), most likely because of an excess of free EBI3 over EBI3/p28 heterodimer (see below) and low sensitivity of p28 blot.

To confirm that placental cells produce IL-27, the amount of IL-27 present in the culture supernatants of term placenta explants harvested at various times of culture was measured by ELISA. As shown on FIG. 2B, concentrations ranging from ˜1 to 4 ng/ml of IL-27 were detected, confirming that placental cells produce IL-27. These supernatants had been previously tested for EBI3 by ELISA, and contained from 10 to 33 ng/ml of EBI3 (see, e.g., Devergne, et al. (2001) Am J Pathol 159:1763-1776). Thus, in all cases, an excess of free EBI3 over IL-27 heterodimer (˜10-fold excess on average) was present. A similar finding was observed in the culture supernatants from activated human monocyte-derived dendritic cells, which contained amounts of EBI3 and IL-27 in the range of those observed in the present experiments (see, e.g., Nagai et al. (2003) J Immunol 17:5233-5243). This excess of EBI3 production is also consistent with previous studies involving IL-27 expression in human placenta (see, e.g., Pflanz, et al. (2002) supra).

It has been previously shown that increased levels of EBI3 were present in the sera of pregnant women. EBI3 serum levels were already upregulated after two months of pregnancy and gradually increased with gestional age to reach concentrations of up to 400 ng/ml at the end of pregnancy (see, e.g., Devergne, et al. (2001) supra). However, when sera from 7 pregnant women were tested for IL-27 by ELISA, no significant levels of IL-27 were detected, regardless of the term. This lack of detection of IL-27 in the sera may be due to a low half-life of IL-27 in vivo. Indeed, it should be noted that in contrast to other heterodimeric cytokines such as IL-12 and IL-23 whose subunits are covalently linked, the association between EBI3 and p28 is not covalent. As a consequence, the heterodimer may tend to dissociate in vivo, thereby lowering its detection in the serum.

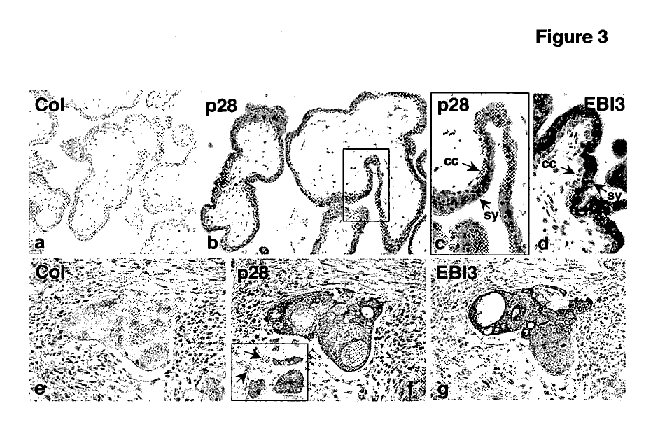

Placental floating villi are composed of an inner layer of cytotrophoblasts and an outer layer of syncytiotrophoblasts. Previously, we showed by immunohistochemistry that EBI3 expression in floating villi was restricted to syncytiotrophoblasts. These cells were positive for EBI3 throughout pregnancy, whereas villous cytotrophoblasts, Hofbauer cells (placental macrophages) and fetal endothelial cells were negative for EBI3 (Devergne et al. (2001) Am J Pathol 159:1763-1776). To investigate whether syncytiotrophoblasts express the second subunit of IL-27, p28 expression was analyzed by immunohistochemistry with rabbit polyclonal or rat monoclonal anti-p28 Abs in 19 placentae of first, second and third trimester. These placentae, either had been previously tested for EBI3 by immunohistochemistry (see, e.g., Devergne et al. (2001) supra), or if not, were tested in parallel for EBI3 and p28. As shown on FIG. 3 a-c, syncytiotrophoblasts displayed significant p28 staining, whereas no or very low p28 signal was observed in cytotrophoblasts. p28 staining of syncytiotrophoblasts was usually the strongest in first-trimester placentae, but was also observed in second- and third-trimester placentae.

Besides pregnancy, trophoblastic cells can be observed in certain germ cell tumors. Thus, to further analyze the capacity of trophoblast cells to co-express both subunits of IL-27, 7 cases of testicular germ cell tumors, including choriocarcinomas, were analyzed for EBI3 and p28 expression by immunohistochemistry. Similar to placental trophoblast cells, syncytiotrophoblastic cells, but not cytotrophoblastic cells, co-expressed EBI3 and p28 in these tumors (FIG. 3 e-g).

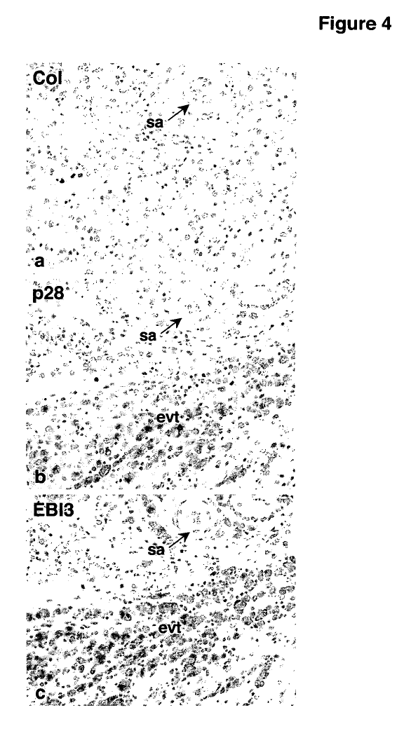

Cytotrophoblasts from placental anchoring villi invade the uterus where they differentiate into extravillous trophoblasts. These invasive extravillous trophoblasts comprise a heterogeneous population of interstitial trophoblasts, multinucleated giant cells and trophoblasts that invade and transform maternal uterine spiral arteries, called endovascular trophoblasts. Previously, it was shown that EBI3 was expressed by interstitial trophoblasts, multinucleated giant cells and trophoblasts invading the wall of spiral arteries, but was downregulated once these cells had differentiated into endovascular trophoblasts. EBI3-positive extravillous trophoblasts were observed throughout pregnancy and constituted the principal source of EBI3 in the decidua, since other cell types present at this site, such as decidual cells and infiltrating lymphocytes, were negative for EBI3 (see, e.g., Devergne et al. (2001) supra). Immunohistochemical studies of p28 expression in the 19 placentae described above showed that extravillous trophoblasts (interstitial trophoblasts and multinucleated giant cells) expressed p28, regardless of the term (FIG. 4). Thus, extravillous trophoblasts, like syncytiotrophoblasts, can co-express both subunits of IL-27.

This work extends previous observations of EBI3 expression at the fetal-maternal interface. The present studies provide evidence that not only EBI3, but also IL-27, is expressed at this site by syncytiotrophoblasts and extravillous trophoblasts. The in situ detection of both subunits of IL-27 by trophoblast cells was not fully expected, since previous in situ analyses failed to detect p28 expression in cells that strongly expressed EBI3, such as dendritic cells or tumoral cells of lymphomas (Larousserie, et al. (2004) supra; Larousserie et al. (2005) supra; and Larousserie et al. (2006) supra.). Expression of IL-27 at the fetal-maternal interface was also not anticipated given the first described functions of this new cytokine. Indeed, initial studies identified IL-27 as a pro-Th1 and pro-inflammatory cytokine, in part because of its ability to confer IL-12 responsiveness to naive CD4⁺ T cells and to increase the expression of IFN-γ and pro-inflammatory cytokines in naive CD4⁺ T cells and monocytes, respectively (see, e.g., Pflanz et al. (2002) supra; and Pflanz et al. (2004) supra).

These findings contrast with the concept that has prevailed over the last decade which stipulated that successful pregnancy was predominantly a Th2 situation, whereas Th1 cytokines were considered to be deletorious to pregnancy (Wegmann et al. (1993) Immunol Today 14:353-356). However, this postulate was later on questioned, since classical Th1 cytokines, such as IFN-γ, IL-12 and IL-18, were shown to be expressed in normal uterus and were suggested to play a positive regulatory role in pregnancy, when expressed at adequate doses (Chaouat et al. (2004) Immunol Lett 92:207-214). Pro-inflammatory cytokines, such as LIF and IL-11, were demonstrated to play a critical role in the implantation process, notably by inducing adhesion molecules on trophoblasts and decidual cells (Chaouat, et al. (2004) supra). These findings led to reconsideration of the Th1/Th2 paradigm and a view of a positive role of Th1 and pro-inflammatory cytokines in pregnancy. In addition, recent studies have shown that IL-27 biological activities extended far beyond its pro-Th1 activity, and based on murine studies a role of IL-27 in dampening excess T cell activity and inflammatory responses was proposed (Trinchieri, et al. (2003) supra). Recently, the anti-inflammatory role of IL-27 was partially elucidated by the demonstration that IL-27 inhibits the development of Th17 cells, the major pro-inflammatory Th cell subset (Batten et al. (2006) Nat Immunol 7:929-936; and Stumhofer et al. (2006) Nat Immunol 7:937-945).

Taken together, these observations led to a model in which IL-27, depending on the context and the cytokine milieu, may play distinct, opposite, roles in the regulation of Th and inflammatory responses. The prevalence of its immunosuppressive functions over its pro-Th1 activity was suggested in many mouse models (see, e.g., Batten et al. (2006) supra; Stumhofer et al. (2006) supra; Villarino et al. (2003) Immunity 19:645-655; Hamano et al. (2003) Immunity 19:657-667; and Wirtz et al. (2006) J Exp Med 203:1875-1881).

By RT-PCR analysis, it was shown that genes encoding IL-27 receptor chains were expressed in the placenta at various gestational ages. Because of the lack of an IL-27R antibody suitable for immunohistochemisrty, the precise nature of the cells expressing IL-27 receptor in situ could not be established. However, previous studies have shown that this receptor is expressed by numerous cell types, many of which such as NK cells, NKT cells, CD8⁺T cells and CD4⁺CD25⁺ regulatory T cells (VIIIarino (2005) J Immunol 174:7684-7691), are present at the fetal-maternal interface (Boyson et al. (2002) Proc Natl Acad Sci USA 99:13741-13746; Shao et al. (2005) J Immunol 174:7539-7547; Sasaki et al. (2004) Mol Hum Reprod 10:347-353; Somerset et al. (2004) Immunology 112:38-43; Tilburgs et al. (2006) Placenta 27 Suppl A:S47-53; and Zenclussen (2006) Springer Semin Immun 28:31-39). Among these, decidual NK cells are the most abundant lymphocyte population infiltrating the decidua, particularly during the first and second trimester of gestation, when they account for over 50% of the leukocyte population. Decidual NK cells and invasive trophoblasts directly interact and mutually regulate their functions, in part through cytokine production (Moffett-King (2002) Nat Rev Immunol 2:656-663; Parham (2004) J Exp Med 200:951-955; Tabiasco et al. (2006) Placenta 27 Suppl A:S34-9; and Le Bouteiller and Tabiasco (2006) Nat Med 12:991-992). Regulated interactions between these two cell types have notably been shown to play a critical role in vascular transformation of maternal uterine arteries, a key event necessary to enlarge vessels and provide sufficient blood supply to the developing fetoplacental unit. Uterine NK cells display pro-angiogenic activity via the production of pro-angiogenic factors, such as VEGF (Hanna, et al. (2006) Nat Med 12:1065-1074). Interestingly, these cells also produce IFN-γ which, despite its abortive effect at high doses, is necessary for proper development of the decidua and uterine vascular modification during normal murine pregnancy (Ashkar et al. (2000) J Exp Med 192:259-269). It has been shown that IL-27 is involved in IFN-γ production by peripheral NK cells (Pflanz, et al. (2002) supra), and may also regulate the production of IFN-γ and possibly other soluble factors, by decidual NK cells. Similarly, IL-27 may modulate cytokine production by decidual NKT cells, as suggested from previous mouse studies (Yamanaka et al. (2004) J Immunol 172:3590-3596).

In addition to IFN-γ, IL-12 and IL-18 have also been suggested to play a role in angiogenesis during pregnancy. Thus, in women with repeated implantation failure after in vitro fertilization, dysregulated IL-12 and IL-18 expression profiles (absence or excess) were observed in the endometrium, in association with vascular alterations (Ledée-Bataille et al. (2004) Fertil Steril 81:59-65). Recently, IL-27 has been shown to act directly on endothelial cells and to inhibit angiogenesis by inducing production of antiangiogenic chemokines IP-10 and MIG (Shimizu, et al. (2006) supra). Therefore, IL-27 production by extravillous trophoblasts may provide a negative feedback mechanism to prevent excess pro-angiogenic activity. Indeed, extravillous trophoblasts are already known to display anti-angiogenic activity by inducing apoptosis of endothelial cells through Fas/FasL interaction (Ashton, et al. (2005) Arterioscler Thromb Vasc Biol 25:102-108) or through the secretion of soluble HLA-G1 (Fons, et al. (2006) supra).

In addition to its role in angiogenesis, IL-27 may be involved locally in the suppression of T-cell responses or in the regulation of inflammatory responses in normal or pathological conditions. Although production of IL-17 in the decidua has been evidenced in human and mouse (Ostojic et al. (2003) Am J Reprod Immunol 49:101-112) it remains to be determined whether Th17 cells are present in human decidua.

Finally, it should be noted that, although IL-27 production was detected in the placenta, its production is far less abundant than that of EBI3. It has been suggested, but not demonstrated yet, that EBI3 may play a role independently from its association with p28, possibly as an IL-27 antagonist. If so, this excess of free EBI3 may regulate IL-27 function.

### III. IL-27 AGONISTS

Agonism of the activities of IL-27 can be achieved by an IL-27 agonist, e.g., IL-27 polypeptide or mutant thereof, an agonist antibody or fragment thereof, raised against the ligand, IL-27, an agonist antibody to a subunit of the ligand, e.g., anti-p28 antibody or anti-EBI3 antibody, an antibody that binds to both p28 and EBI3, that may increase the ligand interaction with the receptor complex. The antibody may also be an agonist antibody to the IL-27 receptor complex (WSX-1/TCCR, gp130). The agonist antibody will mimic binding of the ligand to the receptor and induce signaling of the receptor complex.

Alternatively, small molecule libraries may be screened for compounds which may mimic or enhance the interaction or signaling mediated by an identified ligand-receptor pairing.

The present invention provides for the use of an antibody or binding composition which specifically binds to a specified cytokine ligand, preferably mammalian, e.g., primate, human, cat, dog, rat, or mouse. Antibodies can be raised to various cytokine proteins, including individual, polymorphic, allelic, strain, or species variants, and fragments thereof, both in their naturally occurring (full-length) forms or in their recombinant forms. Additionally, antibodies can be raised to receptor proteins in both their native (or active) forms or in their inactive, e.g., denatured, forms. Anti-idiotypic antibodies may also be used.

A number of immunogens may be selected to produce antibodies specifically reactive with ligand or receptor proteins. Recombinant protein is a preferred immunogen for the production of monoclonal or polyclonal antibodies. Naturally occurring protein, from appropriate sources, e.g., primate, rodent, etc., may also be used either in pure or impure form. Synthetic peptides, made using the appropriate protein sequences, may also be used as an immunogen for the production of antibodies. Recombinant protein can be expressed and purified in eukaryotic or prokaryotic cells as described, e.g., in Coligan, et al. (eds.) (1995 and periodic supplements) Current Protocols in Protein Science, John Wiley & Sons, New York, N.Y.; and Ausubel, et al (eds.) (1987 and periodic supplements) Current Protocols in Molecular Biology, Greene/Wiley, New York, N.Y. Naturally folded or denatured material can be used, as appropriate, for producing antibodies. Either monoclonal or polyclonal antibodies may be generated, e.g., for subsequent use in immunoassays to measure the protein, or for immunopurification methods.

Methods of producing polyclonal antibodies are well known to those of skill in the art. Typically, an immunogen, preferably a purified protein, is mixed with an adjuvant and animals are immunized with the mixture. The animal's immune response to the immunogen preparation is monitored by taking test bleeds and determining the titer of reactivity to the protein of interest. For example, when appropriately high titers of antibody to the immunogen are obtained, usually after repeated immunizations, blood is collected from the animal and antisera are prepared. Further fractionation of the antisera to enrich for antibodies reactive to the protein can be performed if desired. See, e.g., Harlow and Lane; or Coligan. Immunization can also be performed through other methods, e.g., DNA vector immunization. See, e.g., Wang, et al. (1997) Virology 228:278-284.

Monoclonal antibodies may be obtained by various techniques familiar to researchers skilled in the art. Typically, spleen cells from an animal immunized with a desired antigen are immortalized, commonly by fusion with a myeloma cell. See, Kohler and Milstein (1976) Eur. J. Immunol. 6:511-519. Alternative methods of immortalization include transformation with Epstein Barr Virus, oncogenes, or retroviruses, or other methods known in the art. See, e.g., Doyle, et al. (eds.) (1994 and periodic supplements) Cell and Tissue Culture: Laboratory Procedures, John Wiley and Sons, New York, N.Y. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according, e.g., to the general protocol outlined by Huse, et al. (1989) Science 246:1275-1281.

Antibodies or binding compositions, including binding fragments and single chain versions, against predetermined fragments of ligand or receptor proteins can be raised by immunization of animals with conjugates of the fragments with carrier proteins. Monoclonal antibodies are prepared from cells secreting the desired antibody. These antibodies can be screened for binding to normal or defective protein. Antibodies or binding compositions will usually bind with at least a K_{D} of about 10⁻³ M, more usually at least 10⁻⁶ M, typically at least 10⁻⁷ M, more typically at least 10⁻⁸ M, preferably at least about 10⁻⁹ M, and more preferably at least 10⁻¹⁰ M, and most preferably at least 10⁻¹¹ M (see, e.g., Presta, et al. (2001) Thromb. Haemost. 85:379-389; Yang, et al. (2001) Crit. Rev. Oncol. Hematol. 38:17-23; Carnahan, et al. (2003) Clin. Cancer Res. (Suppl.) 9:3982s-3990s).

In some instances, it is desirable to prepare monoclonal antibodies (mAbs) from various mammalian hosts, such as mice, rodents, primates, humans, etc. Description of techniques for preparing such monoclonal antibodies may be found in, e.g., Stites, et al. (eds.) Basic and Clinical Immunology (4th ed.) Lange Medical Publications, Los Altos, Calif., and references cited therein; Harlow and Lane (1988) Antibodies: A Laboratory Manual, CSH Press; Goding (1986) Monoclonal Antibodies: Principles and Practice (2d ed.) Academic Press, New York, N.Y.; and particularly in Kohler and Milstein (1975) Nature 256:495-497, which discusses one method of generating monoclonal antibodies. Summarized briefly, this method involves injecting an animal with an immunogen. The animal is then sacrificed and cells taken from its spleen, which are then fused with myeloma cells. The result is a hybrid cell or “hybridoma” that is capable of reproducing in vitro. The population of hybridomas is then screened to isolate individual clones, each of which secrete a single antibody species to the immunogen. In this manner, the individual antibody species obtained are the products of immortalized and cloned single B cells from the immune animal generated in response to a specific site recognized on the immunogenic substance.

The polypeptides and antibodies of the present invention may be used with or without modification, including chimeric or humanized antibodies. Frequently, the polypeptides and antibodies will be labeled by joining, either covalently or non-covalently, a substance which provides for a detectable signal. A wide variety of labels and conjugation techniques are known and are reported extensively in both the scientific and patent literature. Suitable labels include radionuclides, enzymes, substrates, cofactors, inhibitors, fluorescent moieties, chemiluminescent moieties, magnetic particles, and the like. Patents teaching the use of such labels include U.S. Pat. Nos. 3,817,837; 3,850,752; 3,939,350; 3,996,345; 4,277,437; 4,275,149; and 4,366,241. Also, recombinant immunoglobulins may be produced, see, Cabilly, U.S. Pat. No. 4,816,567, or as described below; and Queen, et al. (1989) Proc. Natl. Acad. Sci. USA 86:10029-10033; or made in transgenic mice, see Mendez, et al. (1997) Nature Genetics 15:146-156; also see Abgenix and Medarex technologies.

Monoclonal antibodies are generally derived from non-human sources, rather than from human sources (Harlow and Lane (1988) Antibodies A Laboratory Manual, Cold Spring Harbor Laboratory, Cold Spring Harbor, N.Y., pp. 139-243). The use of non-human sources can limit the therapeutic efficiency of a monoclonal antibody. Antibodies derived from murine or other non-human sources can have the undesired properties of provoking an immune response, weak recruitment of effector function, and rapid clearance from the bloodstream (Baca, et al. (1997) J. Biol. Chem. 272:10678-10684). For these reasons, it may be desired to prepare therapeutic antibodies by humanization.

“Humanized antibody” means an antibody comprising an antigen-binding region of nonhuman origin, e.g., rodent, and at least a portion of an immunoglobulin of human origin, e.g., a human framework region, a human constant region, or portion thereof (see, e.g., U.S. Pat. No. 6,352,832).

A humanized antibody contains the amino acid sequences from six complementarity determining regions (CDRs) of the parent mouse antibody, which are grafted on a human antibody framework. The content of non-human sequence in humanized antibodies is preferably low, i.e., about 5% (Baca, et al. (1997) J. Biol. Chem. 272:10678-10684). To achieve optimal binding, the humanized antibody may need fine-tuning, by changing certain framework amino acids, usually involved in supporting the conformation of the CDRs, back to the corresponding amino acid found in the parent mouse antibody. The framework amino acids that are generally changed back to those of the parent are those involved in supporting the conformation of the CDR loops (Chothia, et al. (1989) Nature 342:877-883; Foote and Winter (1992) J. Mol. Biol. 224:487-499). The framework residues that most often influence antigen binding is relatively small, and may be a small as eleven residues (Baca, et al. (1997) J. Biol. Chem. 272:10678-10684).

Humanized antibodies include antibodies having all types of constant regions, including IgM, IgG, IgD, IgA and IgE, and any isotype, including IgG1, IgG2, IgG3 and IgG4. When it is desired that the humanized antibody exhibit cytotoxic activity, the constant domain is usually a complement-fixing constant domain and the class is typically IgG1. When such cytotoxic activity is not desirable, the constant domain can be of the IgG2 class. The humanized antibody may comprise sequences from more than one class or isotype (U.S. Pat. No. 6,329,511 issued to Vasquez, et al.). The phage display technique can be used for screening for and selecting antibodies with high binding affinity (Hoogenboom and Chames (2000) Immunol. Today 21:371-377; Barbas, et al. (2001) Phage Display:A Laboratory Manual, Cold Spring Harbor Laboratory, Cold Spring Harbor, N.Y.; Kay, et al. (1996) Phage Display of Peptides and Proteins:A Laboratory Manual, Academic Press, San Diego, Calif.).

Antibodies can also be prepared or designed using the phage display method or human antibody libraries contained in transgenic mice (see, e.g., de Bruin, et al. (1999) Nature Biotechnology 17:397-399; Vaughan, et al. (1996) Nature Biotechnology 14:309-314; Barbas (1995) Nature Medicine 1:837-839; Mendez, et al. (1997) Nature Genetics 15:146-156; Huse, et al. (1989) Science 246:1275-1281; Ward, et al. (1989) Nature 341:544-546).

Antibodies are merely one form of specific binding compositions. Other binding compositions, which will often have similar uses, include molecules that bind with specificity to ligand or receptor, e.g., in a binding partner-binding partner fashion, an antibody-antigen interaction, or in a natural physiologically relevant protein-protein interaction, either covalent or non-covalent, e.g., proteins which specifically associate with desired protein. The molecule may be a polymer, or chemical reagent. A functional analog may be a protein with structural modifications, or may be a structurally unrelated molecule, e.g., which has a molecular shape which interacts with the appropriate binding determinants.

### IV. IL-27 POLYPEPTIDE AGONISTS

IL-27 agonist polypeptides of the present invention can be expressed in various cell lines. In these embodiments, sequences encoding the IL-27 heterodimer, or single subunits thereof (e.g., EBI-3 or p28) can be used for transformation of a suitable mammalian host cell. According to these embodiments, transformation can be achieved using any known method for introducing polynucleotides into a host cell, including, for example packaging the polynucleotide in a virus (or into a viral vector) and transducing a host cell with the virus (or vector) or by transfection procedures known in the art. Such procedures are exemplified by U.S. Pat. Nos. 4,399,216, 4,912,040, 4,740,461, and 4,959,455 (all of which are hereby incorporated herein by reference for any purpose). Generally, the transformation procedure used may depend upon the host to be transformed. Methods for introducing heterologous polynucleotides into mammalian cells are well known in the art and include, but are not limited to, dextran-mediated transfection, calcium phosphate precipitation, polybrene mediated transfection, protoplast fusion, electroporation, encapsulation of the polynucleotide(s) in liposomes, and direct microinjection of the DNA into nuclei.

According to certain embodiments of the methods of the invention, a nucleic acid molecule encoding the IL-27 heterodimer or individual subunits thereof, is inserted into an appropriate expression vector using standard ligation techniques. The vector is typically selected to be functional in the particular host cell employed (i.e., the vector is compatible with the host cell machinery such that amplification of the gene and/or expression of the gene can occur). For a review of expression vectors, see, Goeddel (ed.), 1990, Meth. Enzymol. Vol. 185, Academic Press. N.Y.

Typically, expression vectors used in any of the host cells will contain sequences for plasmid maintenance and for cloning and expression of exogenous nucleotide sequences. Such sequences, collectively referred to as “flanking sequences” in certain embodiments will typically include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Each of these sequences is discussed below.

Optionally, the vector may contain a “tag”-encoding sequence, i.e., an oligonucleotide molecule located at the 5′ or 3′ end of the polypeptide coding sequence; the oligonucleotide sequence encodes polyHis (such as hexaHis), or another “tag” such as FLAG, HA (hemaglutinin influenza virus), or myc for which commercially available antibodies exist. This tag is typically fused to the polypeptide upon expression of the polypeptide, and can serve as a means for affinity purification or detection of the antibody from the host cell. Affinity purification can be accomplished, for example, by column chromatography using antibodies against the tag as an affinity matrix. Optionally, the tag can subsequently be removed from the purified polypeptide by various means such as using certain peptidases for cleavage.

Flanking sequences may be homologous (i.e., from the same species and/or strain as the host cell), heterologous (i.e., from a species other than the host cell species or strain), hybrid (i.e., a combination of flanking sequences from more than one source), synthetic or native. As such, the source of a flanking sequence may be any prokaryotic or eukaryotic organism, any vertebrate or invertebrate organism, or any plant, provided that the flanking sequence is functional in, and can be activated by, the host cell machinery.

Flanking sequences useful in the vectors of this invention may be obtained by any of several methods well known in the art. Typically, flanking sequences useful herein will have been previously identified by mapping and/or by restriction endonuclease digestion and can thus be isolated from the proper tissue source using the appropriate restriction endonucleases. In some cases, the full nucleotide sequence of a flanking sequence may be known. Here, the flanking sequence may be synthesized using the methods described herein for nucleic acid synthesis or cloning.

Where all or only a portion of the flanking sequence is known, it may be obtained using polymerase chain reaction (PCR) and/or by screening a genomic library with a suitable probe such as an oligonucleotide and/or flanking sequence fragment from the same or another species. Where the flanking sequence is not known, a fragment of DNA containing a flanking sequence may be isolated from a larger piece of DNA that may contain, for example, a coding sequence or even another gene or genes. Isolation may be accomplished by restriction endonuclease digestion to produce the proper DNA fragment followed by isolation using agarose gel purification, Qiagen® column chromatography (Chatsworth, Calif.), or other methods known to the skilled artisan. The selection of suitable enzymes to accomplish this purpose will be readily apparent to one of ordinary skill in the art.

An origin of replication is typically a part of those prokaryotic expression vectors purchased commercially, and the origin aids in the amplification of the vector in a host cell. If the vector of choice does not contain an origin of replication site, one may be chemically synthesized based on a known sequence, and ligated into the vector. For example, the origin of replication from the plasmid pBR322 (New England Biolabs, Beverly, Mass.) is suitable for most gram-negative bacteria and various viral origins (e.g., SV40, polyoma, adenovirus, vesicular stomatitus virus (VSV), or papillomaviruses such as HPV or BPV) are useful for cloning vectors in mammalian cells. Generally, the origin of replication component is not needed for mammalian expression vectors (for example, the SV40 origin is often used only because it also contains the virus early promoter).

A transcription termination sequence is typically located 3′ to the end of a polypeptide coding region and serves to terminate transcription. Usually, a transcription termination sequence in prokaryotic cells is a G-C rich fragment followed by a poly-T sequence. While the sequence is easily cloned from a library or even purchased commercially as part of a vector, it can also be readily synthesized using methods for nucleic acid synthesis such as those described herein.

A selectable marker gene encodes a protein necessary for the survival and growth of a host cell grown in a selective culture medium. Typical selection marker genes encode proteins that (a) confer resistance to antibiotics or other toxins, e.g., ampicillin, tetracycline, or kanamycin for prokaryotic host cells; (b) complement auxotrophic deficiencies of the cell; or (c) supply critical nutrients not available from complex or defined media. Preferred selectable markers are the kanamycin resistance gene, the ampicillin resistance gene, and the tetracycline resistance gene. A neomycin resistance gene may also be used for selection in both prokaryotic and eukaryotic host cells.

Other selectable genes may be used to amplify the gene that will be expressed. Amplification is the process wherein genes that are in greater demand for the production of a protein critical for growth or cell survival are reiterated generally in tandem within the chromosomes of successive generations of recombinant cells. Examples of suitable selectable markers for mammalian cells include dihydrofolate reductase (DHFR) and promoterless thymidine kinase. Mammalian cell transformants are placed under selection pressure wherein only the transformants are uniquely adapted to survive by virtue of the selectable gene present in the vector. Selection pressure is imposed by culturing the transformed cells under conditions in which the concentration of selection agent in the medium is successively increased, thereby leading to the amplification of both the selectable gene and the DNA that encodes another gene. As a result, increased quantities of a polypeptide can be synthesized from the amplified DNA.

A ribosome-binding site is usually necessary for translation initiation of mRNA and is characterized by a Shine-Dalgarno sequence (prokaryotes) or a Kozak sequence (eukaryotes). The element is typically located 3′ to the promoter and 5′ to the coding sequence of the polypeptide to be expressed.

In some cases, such as where glycosylation is desired in a eukaryotic host cell expression system, one may manipulate the various pre- or prosequences to improve glycosylation or yield. For example, one may alter the peptidase cleavage site of a particular signal peptide, or add pro-sequences, which also may affect glycosylation. The final protein product may have, in the −1 position (relative to the first amino acid of the mature protein) one or more additional amino acids incident to expression, which may not have been totally removed. For example, the final protein product may have one or two amino acid residues found in the peptidase cleavage site, attached to the amino-terminus. Alternatively, use of some enzyme cleavage sites may result in a slightly truncated form of the desired polypeptide, if the enzyme cuts at such area within the mature polypeptide.

The expression and cloning vectors of the invention will typically contain a promoter that is recognized by the host organism and operably linked to the molecule encoding the antibody. Promoters are untranscribed sequences located upstream (i.e., 5′) to the start codon of a structural gene (generally within about 100 to 1000 bp) that control the transcription of the structural gene. Promoters are conventionally grouped into one of two classes: inducible promoters and constitutive promoters. Inducible promoters initiate increased levels of transcription from DNA under their control in response to some change in culture conditions, such as the presence or absence of a nutrient or a change in temperature. Constitutive promoters, on the other hand, initiate continual gene product production; that is, there is little or no control over gene expression. A large number of promoters, recognized by a variety of potential host cells, are well known. A suitable promoter is operably linked to the DNA encoding heavy chain or light chain comprising an antibody of the invention by removing the promoter from the source DNA by restriction enzyme digestion and inserting the desired promoter sequence into the vector.

Suitable promoters for use with yeast hosts are also well known in the art. Yeast enhancers are advantageously used with yeast promoters. Suitable promoters for use with mammalian host cells are well known and include, but are not limited to, those obtained from the genomes of viruses such as polyoma virus, fowlpox virus, adenovirus (such as Adenovirus 2), bovine papilloma virus, avian sarcoma virus, cytomegalovirus, retroviruses, hepatitis-B virus and most preferably Simian Virus 40 (SV40). Other suitable mammalian promoters include heterologous mammalian promoters, for example, heat-shock promoters and the actin promoter.

Additional promoters which may be of interest include, but are not limited to: the SV40 early promoter region (Bemoist and Chambon, 1981, Nature 290:304-10); the CMV promoter; the promoter contained in the 3′ long terminal repeat of Rous sarcoma virus (Yamamoto et al., 1980, Cell 22:787-97); the herpes thymidine kinase promoter (Wagner et al., 1981, Proc. Natl. Acad. Sci. USA 78:1444-45); the regulatory sequences of the metallothionine gene (Brinster et al., 1982, Nature 296:39-42); prokaryotic expression vectors such as the beta-lactamase promoter (VIIIa-Kamaroff et al., 1978, Proc. Natl. Acad. Sci. USA 75:3727-31); or the tac promoter (DeBoer et al., 1983, Proc. Natl. Acad. Sci. USA 80:21-25). Also of interest are the following animal transcriptional control regions, which exhibit tissue specificity and have been utilized in transgenic animals: the elastase I gene control region that is active in pancreatic acinar cells (Swift et al., 1984, Cell 38:639-46; Omitz et al., 1986, Cold Spring Harbor Symp. Quant. Biol. 50:399-409 (1986); MacDonald, 1987, Hepatology 7:425-515); the insulin gene control region that is active in pancreatic beta cells (Hanahan, 1985, Nature 315:115-22); the immunoglobulin gene control region that is active in lymphoid cells (Grosschedl et al., 1984, Cell 38:647-58; Adames et al., 1985, Nature 318:533-38; Alexander et al., 1987, Mol. Cell. Biol. 7:1436-44); the mouse mammary tumor virus control region that is active in testicular, breast, lymphoid and mast cells (Leder et al., 1986, Cell 45:485-95); the albumin gene control region that is active in liver (Pinkert et al., 1987, Genes and Devel. 1:268-76); the alpha-feto-protein gene control region that is active in liver (Krumlauf et al., 1985, Mol. Cell. Biol. 5:1639-48; Hammer et al., 1987, Science 235:53-58); the alpha 1-antitrypsin gene control region that is active in liver (Kelsey et al., 1987, Genes and Delia 1:161-71); the beta-globin gene control region that is active in myeloid cells (Mogram et al., 1985, Nature 315:338-40; Kollias et al., 1986, Cell 46:89-94); the myelin basic protein gene control region that is active in oligodendrocyte cells in the brain (Readhead et al., 1987, Cell 48:703-12); the myosin light chain-2 gene control region that is active in skeletal muscle (Sani, 1985, Nature 314:283-86); and the gonadotropic releasing hormone gene control region that is active in the hypothalamus (Mason et al., 1986, Science 234:1372-78).

An enhancer sequence may be inserted into the vector to increase transcription of DNA encoding light chain or heavy chain comprising an antibody of the invention by higher eukaryotes. Enhancers are cis-acting elements of DNA, usually about 10-300 by in length, that act on the promoter to increase transcription. Enhancers are relatively orientation- and position-independent. They have been found 5′ and 3′ to the transcription unit. Several enhancer sequences available from mammalian genes (e.g., globin, elastase, albumin, alpha-feto-protein and insulin) are known. Typically, however, an enhancer from a virus is used. The SV40 enhancer, the cytomegalovirus early promoter enhancer, the polyoma enhancer, and adenovirus enhancers known in the art are exemplary enhancing elements for the activation of eukaryotic promoters. While an enhancer may be spliced into the vector at a position 5′ or 3′ to a nucleic acid molecule, it is typically located at a site 5′ from the promoter.

Expression vectors of the invention may be constructed from a starting vector such as a commercially available vector. Such vectors may or may not contain all of the desired flanking sequences. Where one or more of the flanking sequences described herein are not already present in the vector, they may be individually obtained and ligated into the vector. Methods used for obtaining each of the flanking sequences are well known to one skilled in the art.

After the vector has been constructed and a nucleic acid molecule encoding light chain or heavy chain or light chain and heavy chain has been inserted into the proper site of the vector, the completed vector may be inserted into a suitable host cell for amplification and/or polypeptide expression. The transformation of an expression vector for an antibody into a selected host cell may be accomplished by well known methods including transfection, infection, calcium phosphate co-precipitation, electroporation, microinjection, lipofection, DEAE-dextran mediated transfection, or other known techniques. The method selected will in part be a function of the type of host cell to be used. These methods and other suitable methods are well known to the skilled artisan, and are set forth, for example, in Sambrook et al., supra.

The host cell, when cultured under appropriate conditions, synthesizes an antibody that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule.

Mammalian cell lines available as hosts for expression are well known in the art and include, but are not limited to, many immortalized cell lines available from the American Type Culture Collection (A.T.C.C.), including but not limited to Chinese hamster ovary (CHO) cells, HeLa cells, baby hamster kidney (BHK) cells, monkey kidney cells (COS), human hepatocellular carcinoma cells (e.g., Hep G2), and a number of other cell lines.

### V. THERAPEUTIC COMPOSITIONS

The present invention provides methods for using agonists of IL-27 for the treatment of, e.g., reproductive disorders, including recurring miscarriages, improper embryonic implantation, pre-eclampsia, etc.

The antagonists and/or agonists of the present invention can be administered alone or in combination with another agonist of the same or accompanying pathway; or other compounds used for the treatment of symptoms. Diagnostic methods include such aspects as prediction of prognosis; definition of subsets of patients who will either respond or not respond to a particular therapeutic course; diagnosis of reproductive related disorders or subtypes of this disorder; or assessing response to therapy.

Treatment, therapy, or diagnosis can be effected by direct administration of the agonist or antagonist or by administration of a nucleic acid encoding the agonist or antagonist. The agonist encompasses a binding composition derived from an antibody, an antibody or antibody fragment that specifically binds IL-27 or an IL-27R, an IL-27 polypeptide or variant, or a vector expressing an nucleic acid encoding the agonist (see, e.g., Arenz and Schepers (2003) Naturwissenschaften 90:345-359; Sazani and Kole (2003) J. Clin. Invest. 112:481-486; Pirollo, et al. (2003) Pharmacol. Therapeutics 99:55-77; Wang, et al. (2003) Antisense Nucl. Acid Drug Devel. 13:169-189).

To prepare pharmaceutical or sterile compositions including the polypeptide or agonist antibody, binding composition thereof, or small molecule agonist, the entity is admixed with a pharmaceutically acceptable carrier or excipient which is preferably inert. Preparation of such pharmaceutical compositions is known in the art, see, e.g., Remington's Pharmaceutical Sciences and U.S. Pharmacopeia: National Formulary, Mack Publishing Company, Easton, Pa. (1984).

Antibodies, binding compositions, or cytokines are normally administered parentally, preferably intravenously. Since such proteins or peptides may be immunogenic they are preferably administered slowly, either by a conventional IV administration set or from a subcutaneous depot, e.g., as taught by Tomasi, et al, U.S. Pat. No. 4,732,863. Methods to minimize immunological reactions may be applied. Small molecule entities may be orally active.

Compositions which are suitable for vaginal administration also include pessaries, tampons, creams, gels, pastes, foams, films, or spray compositions containing such carriers as are known in the art to be appropriate. The carrier employed in the should be compatible with vaginal administration. Combinations can be, e.g., in solid, semi-solid and liquid dosage forms, such as douches, foams, films, ointments, creams, balms, gels, salves, pastes, slurries, vaginal suppositories, or sexual lubricants.

The active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially, e.g., from Alza Corporation and Nova Pharmaceuticals, Inc. Liposomal suspensions (including liposomes targeted to infected cells with monoclonal antibodies to viral antigens) can also be used as pharmaceutically acceptable carriers. These may be prepared according to methods known to those skilled in the art, for example, as described in U.S. Pat. No. 4,522,811.

When administered parenterally the biologics will be formulated in a unit dosage injectable form (solution, suspension, emulsion) in association with a pharmaceutically acceptable parenteral vehicle. Such vehicles are typically inherently nontoxic and nontherapeutic. The therapeutic may be administered in aqueous vehicles such as water, saline, or buffered vehicles with or without various additives and/or diluting agents. Alternatively, a suspension, such as a zinc suspension, can be prepared to include the peptide. Such a suspension can be useful for subcutaneous (SQ) or intramuscular (IM) injection. The proportion of biologic and additive can be varied over a broad range so long as both are present in effective amounts. The antibody is preferably formulated in purified form substantially free of aggregates, other proteins, endotoxins, and the like, at concentrations of about 5 to 30 mg/ml, preferably 10 to 20 mg/ml. Preferably, the endotoxin levels are less than 2.5 EU/ml. See, e.g., Avis, et al. (eds.) (1993) Pharmaceutical Dosage Forms: Parenteral Medications, 2d ed., Dekker, NY; Lieberman, et al. (eds.) (1990) Pharmaceutical Dosage Forms: Tablets, 2d ed., Dekker, NY; Lieberman, et al. (eds.) (1990) Pharmaceutical Dosage Forms: Disperse Systems, Dekker, NY; Fodor, et al. (1991) Science 251:767-773, Coligan (ed.) Current Protocols in Immunology; Hood, et al. (1984) Immunology, Pearson, Upper Saddle River, N.Y.; Paul (ed.) (1999) Fundamental Immunology, 4^{th} ed., Lippincott Williams & Wilkins Publishers, Phila., PA; Parce, et al., (1989) Science 246:243-247; Owicki, et al. (1990) Proc. Natl. Acad. Sci. USA 87:4007-4011; and Blundell and Johnson (1976) Protein Crystallography, Academic Press, New York.

Selecting an administration regimen for a therapeutic depends on several factors, including the serum or tissue turnover rate of the entity, the level of symptoms, the immunogenicity of the entity, and the accessibility of the target cells, timing of administration, etc. Preferably, an administration regimen maximizes the amount of therapeutic delivered to the patient consistent with an acceptable level of side effects. Accordingly, the amount of biologic delivered depends in part on the particular entity and the severity of the condition being treated. Guidance in selecting appropriate antibody doses is found in, e.g., Bach, et al., chapter 22, in Ferrone, et al. (eds.) (1985) Handbook of Monoclonal Antibodies, Noges Publications, Park Ridge, N.J.; and Haber, et al. (eds.) (1977) Antibodies in Human Diagnosis and Therapy, Raven Press, New York, N.Y. (Russell, pgs. 303-357, and Smith, et al., pgs. 365-389). Alternatively, doses of cytokine or small molecules are determined using standard methodologies.

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. Preferably, a biologic that will be used is derived from the same species as the animal targeted for treatment, thereby minimizing a humoral response to the reagent.

The total weekly dose ranges for antibodies or fragments thereof, which specifically bind to ligand or receptor range generally from about 10 μg, more generally from about 100 μg, typically from about 500 μg, more typically from about 1000 μg, preferably from about 5 mg, and more preferably from about 10 mg per kilogram body weight. Generally the range will be less than 100 mg, preferably less than about 50 mg, and more preferably less than about 25 mg per kilogram body weight. Polypeptide or small molecule therapeutics may be used at similar molarities.

The weekly dose ranges for antagonists of cytokine receptor mediated signaling, e.g., antibody or binding fragments, range from about 1 μg, preferably at least about 5 μg, and more preferably at least about 10 μg per kilogram of body weight. Generally, the range will be less than about 1000 μg, preferably less than about 500 μg, and more preferably less than about 100 μg per kilogram of body weight. Dosages are on a schedule which effects the desired treatment and can be periodic over shorter or longer term. In general, ranges will be from at least about 10 μg to about 50 mg, preferably about 100 μg to about 10 mg per kilogram body weight. Cytokine agonists or small molecule therapeutics will typically be used at similar molar amounts, but because they likely have smaller molecular weights, will have lesser weight doses.

The present invention also provides for administration of biologics in combination with known therapies, e.g., hormones, such as estrogen or progestrone which alleviate the symptoms, e.g., associated with inflammation, or antibiotics or anti-infectives. Daily dosages for glucocorticoids will range from at least about 1 mg, generally at least about 2 mg, and preferably at least about 5 mg per day. Generally, the dosage will be less than about 100 mg, typically less than about 50 mg, preferably less than about 20 mg, and more preferably at least about 10 mg per day. In general, the ranges will be from at least about 1 mg to about 100 mg, preferably from about 2 mg to 50 mg per day. The phrase “effective amount” means an amount sufficient to ameliorate a symptom or sign of the medical condition. Typical mammalian hosts will include mice, rats, cats, dogs, and primates, including humans. An effective amount for a particular patient may vary depending on factors such as the condition being treated, the overall health of the patient, the method route and dose of administration and the severity of side affects. When in combination, an effective amount is in ratio to a combination of components and the effect is not limited to individual components alone

An effective amount of therapeutic will decrease the symptoms typically by at least about 10%; usually by at least about 20%; preferably at least about 30%; or more preferably at least about 50%. The present invention provides reagents which will find use in therapeutic applications as described elsewhere herein, e.g., in the general description for treating disorders associated with the indications described above. Berkow (ed.) The Merck Manual of Diagnosis and Therapy, Merck & Co., Rahway, N.J.; Brauwald, et al. (eds.) (2001) Harrison's Principles of Internal Medicine, 15^{th} ed., McGraw-Hill, NY; Gilman, et al. (eds.) (1990) Goodman and Gilman's: The Pharmacological Bases of Therapeutics, 8th Ed., Pergamon Press; Remington's Pharmaceutical Sciences, 17th ed. (1990), Mack Publishing Co., Easton, Pa.; Langer (1990) Science 249:1527-1533; Merck Index, Merck & Co., Rahway, N.J.; and Physician's Desk Reference (PDR); Cotran, et al. (eds.), supra; and Dale and Federman (eds.) (2000) Scientific American Medicine, Healtheon/WebMD, New York, N.Y.

### VI. DIAGNOSTIC METHODS

The present invention provides a method for detecting the presence of IL-27 or IL-27R in a biological sample. The method involves contacting the biological sample with an agent capable of detecting protein or nucleic acid molecules (e.g., mRNA) such that the presence of one or both of these genes is detected in the biological sample. One agent for detecting mRNA is a labeled nucleic acid probe capable of hybridizing specifically to the mRNA of a particular gene. The nucleic acid probe can be, for example, the full-length cDNA, or a portion thereof, such as an oligonucleotide of at least 15, 30, 50, 100, 250 or 500 nucleotides in length and is sufficiently complimentary to specifically hybridize under stringent conditions to the particular mRNA. Probes can be designed using the publicly available sequences from GenBank (e.g., NM_{—}005755 for human EBI-3; NM_{—}145659 for human p28; NM_{—}004843 for human WSX1/TCCR; and NM_{—}175767 for human gp130). Sequences for any of the aforementioned genes from species other than humans may be obtained by searching GenBank using the desired gene name and the name of the desired organism.

mRNA in a sample is determined using the Perkin Elmer Taqman EZ RT-PCR kit (Perkin Elmer). Gene specific primers and probes can be designed using Primer Express software (Perkin Elmer) and the gene sequences described above by Accession number.

An agent for detecting a particular protein (e.g., an adhesion molecule, an inflammatory cytokine, or an immune cell surface antigen) is a labeled or labelable antibody capable of binding to that specific protein. Antibodies can be polyclonal, or more preferably, monoclonal. An intact antibody, or a fragment thereof (e.g., Fab or F(ab′)₂) can be used. The term “labeled or labelable”, with regard to the probe or antibody, is intended to encompass direct labeling of the probe or antibody by coupling (i.e., physically linking) a detectable substance (e.g., ¹²⁵I) to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

A biological sample comprises a sample which has been isolated from a subject and is subjected to a method of the present invention without further processing or manipulation subsequent to its isolation. In another embodiment, the biological sample can be processed or manipulated subsequent to being isolated and prior to being subjected to a method of the invention. For example, a sample can be refrigerated (e.g., stored at 4° C.), frozen (e.g., stored at −20° C., stored at −135° C., frozen in liquid nitrogen, or cryopreserved using any one of many standard cryopreservation techniques known in the art). Furthermore, a sample can be purified subsequent to isolation from a subject and prior to subjecting it to a method of the present invention. As used herein, the term “purified” when used in the context of a biological sample, is intended to indicate that at least one component of the isolated biological sample has been removed from the biological sample such that fewer components, and consequently, purer components, remain following purification. For example, a serum sample can be separated into one or more components using centrifugation techniques known in the art to obtain partially-purified sample preparation. Furthermore, it is possible to purify a biological sample such that substantially only one component remains. For example, a tissue or tumor sample can be purified such that substantially only the protein or mRNA component of the biological sample remains.

It may be desirable to amplify a component of a biological sample such that detection of the component is facilitated. For example, the mRNA component of a biological sample can be amplified (e.g., by RT-PCR) such that detection of mRNA is facilitated. As used herein, the term “RT-PCR” (“reverse transcriptase-polymerase chain reaction”) includes subjecting mRNA to the reverse transcriptase enzyme resulting in the production of DNA which is complementary to the base sequences of the mRNA. Large amounts of selected cDNA can then be produced via the polymerase chain reaction which relies on the action of heat-stable DNA poly erase for its amplification action. Alternative amplification methods include, but are not limited to, self sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), Q-Beta Replicase (Lizardi, P. M. et all, 1988, Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

The detection methods of the present invention can be used to detect protein or nucleic acid molecules in a biological sample in vitro as well as in vivo. For example, in vitro techniques for detection of mRNA include Northern hybridizations and in situ hybridizations. In vitro techniques for detection of protein include enzyme linked immunosorbent assays (ELISAs), Western blots, immunoprecipitation and immunofluorescence.

In order to determine if the level of IL-27 or IL-27R in a biological sample from a test subject is abnormal, the level of IL-27 or IL-27R from the test subject is compared, for example, to the average level of these molecules determined from women who have had normal pregnancies. If the level of those molecules determined in the test subject is lower (i.e., statistically significantly lower) than the levels for normal pregnancies, the test subject is diagnosed as being at risk for developing immune-mediated spontaneous abortion.

For example, a subject having a 10-20% decrease in the level of IL-27 or IL-27R is diagnosed as being at risk. In yet another embodiment, a subject having a 20-30%, 30-40%, 40-50%, 50-100%, 100-200%, 200-400% (e.g., 2-fold to 4-fold), 4-fold to 10-fold, 10-fold to 100-fold, 100-fold or greater decreased levels, for example, when compared to a suitable or appropriate control, is diagnosed as being at risk for immune-mediated spontaneous abortion.

The present invention also comprises kits for detecting the presence of IL-27 or IL-27R in a biological sample. For example, the kit can comprise a labeled or labelable agent capable of detecting mRNA or polypeptide of at least one subunit of the IL-27 heterodimer or the IL-27R complex in a biological sample and a means for determining the amount of mRNA or polypeptide of IL-27 or IL-27R in a biological sample. The agent can be packaged in a suitable container. The kit can further comprise a means for comparing the amount of IL-27 or IL-27R in the sample with a standard (e.g., a chart showing normal and abnormal ranges for IL-27 or IL-27R levels, or a sample of a suitable or appropriate control) and/or can further comprise instructions for using the kit to detect mRNA or polypeptide at least one subunit of the IL-27 heterodimer or the IL-27R complex.

Current models of maternal immunological tolerance of the embryo have centered on two complementary mechanisms: utilization of indirect cytokine and hormonal signaling and the active immunosuppression of maternal lymphocytes. Recently, a number of known immunomodulatory cytokines were described that have been demonstrated to influence maternal tolerance of the embryo. Whereas the Th1 cytokines promoting cellular cytotoxicity (e.g., during an intracellular parasitic infection) such as TNF-α, IFN-γ, IL-2, IL-1, IL-6, IL-12, and RANTES have been demonstrated to increase spontaneous abortion rates in abortion prone mice, the presence of Th2 cytokines such as CSF-1, GM-CSF, IL-10, IL-4, IL-11, TGF and IL-3 which promote humoral immunity, have been shown to correlate with reduced abortion rates (Chaouat et al., 1990, J Reprod Fertil 89, 447-58; Gafter et al., 1997, J Clin Immunol 17, 408-19). Active immunosuppression has also been documented in studies with factors derived from embryonic sources. In the past, some unidentified proteins and anti-paternal blocking antibodies have been reported to confer modest immunosuppression to the embryo (Herrera-Gonzalez and Dresser, 1993, Dev Comp Immunol 17, 1-18). Examples 7-11 indicate that expression levels of numerous cytokines, as well as adhesion molecules and cell-surface antigens, are altered in mice during pathologic pregnancy, but that many can be normalized by treatment with CTLA4Ig. Altered expression levels of these genes, therefore may be used as diagnostic criteria for determining whether a subject is at risk for or developing or suffering from an immune-mediated spontaneous abortion.

It may also be desirable to use the methods of the present invention, as described above, to monitor the progress of treatment of a subject. For example, a subject (e.g., a human) may be undergoing treatment for immune-mediated spontaneous abortion using any one of the methods of treatment described herein. The methods described above for diagnosis may be used to determine if the level of IL-27 or IL-27R has returned to a normal level. Treatment of the subject can then be modified (e.g., increased or decreased) depending on the results.

The broad scope of this invention is best understood with reference to the following examples, which are not intended to limit the inventions to the specific embodiments.

### EXAMPLES

### I. General Methods

Many of the standard methods below are described or referenced, e.g., in Maniatis, et al. (1982) Molecular Cloning, A Laboratory Manual, Cold Spring Harbor Laboratory, Cold Spring Harbor Press, NY; Sambrook, et al. (1989) Molecular Cloning: A Laboratory Manual (2d ed.) Vols. 1-3, CSH Press, NY; Ausubel, et al., Biology, Greene Publishing Associates, Brooklyn, N.Y.; or Ausubel, et al. (1987 and Supplements) Current Protocols in Molecular Biology, Wiley/Greene, NY; Innis, et al. (eds.) (1990) PCR Protocols: A Guide to Methods and Applications, Academic Press, NY. Methods for protein purification include such methods as ammonium sulfate precipitation, column chromatography, electrophoresis, centrifugation, crystallization, and others. See, e.g., Ausubel, et al. (1987 and periodic supplements); Deutscher (1990) “Guide to Protein Purification,” Methods in Enzymology, vol. 182, and other volumes in this series; Coligan, et al. (1995 and supplements) Current Protocols in Protein Science, John Wiley and Sons, New York, N.Y.; P. Matsudaira (ed.) (1993) A Practical Guide to Protein and Peptide Purification for Microsequencing, Academic Press, San Diego, Calif.; and manufacturer's literature on use of protein purification products, e.g., Pharmacia, Piscataway, N.J., or Bio-Rad Laboratories, Hercules, Calif. Combination with recombinant techniques allow fusion to appropriate segments (epitope tags), e.g., to a FLAG sequence or an equivalent which can be fused, e.g., via a protease-removable sequence. See, e.g., Hochuli (1989) Chemische Industrie 12:69-70; Hochuli (1990) “Purification of Recombinant Proteins with Metal Chelate Absorbent” in Setlow (ed.) Genetic Engineering, Principle and Methods 12:87-98, Plenum Press, NY; and Crowe, et al. (1992) QIAexpress: The High Level Expression & Protein Purification System, QIAGEN, Inc., Chatsworth, Calif.

Standard immunological techniques are described, e.g., in Hertzenberg, et al. (eds.) (1996) Weir's Handbook of Experimental Immunology vols. 1-4, Blackwell, Malden, Mass.; Coligan (1991) Current Protocols in Immunology Wiley/Greene, NY; and Methods in Enzymology vols. 70, 73, 74, 84, 92, 93, 108, 116, 121, 132, 150, 162, and 163. Cytokine assays are described, e.g., in Thomson (ed.) (1998) The Cytokine Handbook (3d ed.) Academic Press, San Diego; Mire-Sluis and Thorpe (1998) Cytokines, Academic Press, San Diego, Calif.; Metcalf and Nicola (1995) The Hematopoietic Colony Stimulating Factors, Cambridge Univ. Press, Cambridge, UK; and Aggarwal and Gutterman (1991) Human Cytokines, Blackwell, Malden, Mass.

Assays for vascular biological activities are well known in the art. They will cover angiogenic and angiostatic activities in tumor, or other tissues, e.g., arterial smooth muscle proliferation (see, e.g., Koyoma, et al. (1996) Cell 87:1069-1078), monocyte adhesion to vascular epithelium (see, e.g., McEvoy, et al. (1997) J. Exp. Med. 185:2069-2077; Ross (1993) Nature 362:801-809; Rekhter and Gordon (1995) Am. J. Pathol. 147:668-677; Thyberg, et al. (1990) Atherosclerosis 10:966-990; Gumbiner (1996) Cell 84:345-357.

Assays for neural cell biological activities are described, e.g., in Wouterlood (ed. 1995) Neuroscience Protocols modules 10, Elsevier; Methods in Neurosciences, Academic Press; and Neuromethods Humana Press, Totowa, N.J. Methodology of developmental systems is described, e.g., in Meisami (ed.) Handbook of Human Growth and Developmental Biology, CRC Press; and Chrispeels (ed.) Molecular Techniques and Approaches in Developmental Biology, Interscience.

FACS analyses are described in Melamed, et al. (1990) Flow Cytometry and Sorting, Wiley-Liss, Inc., New York, N.Y.; Shapiro (1988) Practical Flow Cytometry, Liss, New York, N.Y.; and Robinson, et al. (1993) Handbook of Flow Cytometry Methods, Wiley-Liss, New York, N.Y.

### II. Human Tissues

Frozen tissue samples from 8 placentae ranging from 9 to 40 weeks of pregnancy were analyzed by RT-PCR and/or in co-immunoprecipitation experiments. In addition, formalin-fixed paraffin-embedded tissue samples from 19 placentae from first trimester (4-10 weeks, n=11), second trimester (17-27 weeks, n=4) and third trimester (33-39 weeks, n=4) of gestation were analyzed by immunohistochemistry. Placentae were obtained from voluntary pregnancy termination (first-trimester placentae), therapeutic termination, miscarriage, cesarean section or natural delivery (second- and third-trimester placentae). They were selected for the absence of abnormality on macroscopic and histological examination. No lesion indicative of pre-eclampsia and no infection were observed. Paraffin-embedded tissue samples from 7 cases of testicular germ cell tumors (5 cases of mixed germ cell tumors with a choriocarcinoma component, and 2 cases of seminoma with syncytiotrophoblastic cells) were also analyzed.

Frozen tissues, obtained after informed consent of the patient, and fixed tissues, collected for histological examination and diagnostic purposis, were studied in accordance with French ethical guidelines.

### III. RNA Extraction and RT-PCR Analysis

RNA was isolated from frozen tissues or cells by TRIzol extraction, followed by DNAse I digestion and reverse transcription using M-MLV reverse transcriptase and oligo(dT) primer (all reagents from Invitrogen, Cergy-Pontoise, France). For PCR analysis, the following primers were used: EBI3 5′-CCGAGCCAGGTACTACGTCC-3′(sense) and 5-CCAGTCACTCAGTTCCCCGT-3′ (antisense); p28 5′-GTCTCAGCCTGTTGCTGCTT-3′ (sense) and 5′-GAACCTCGGAGAGCAGCTT-3′ (antisense); IL-27R 5′-CCATACCCCTGACCCCTGTTGAGAT-3′ (sense) and CAGAGGTTCCCTGATACCCACACAT-3′ (antisense); gp130 5′-TCTGGGAGTGCTGTTCTGCTT-3′ (sense) and 5′-TGTGCCTTGGAGGAGTGTGA-3′ (antisense); β2-microglobulin 5′-CCAGCAGAGAATGGAAAGTC-3′ (sense) and 5′-GATGCTGCTTACATGTCTCG-3′ (antisense). The thermal cycle profile was as follows: 1 min at 94° C., 30 sec at 55° C. and 20 sec at 72° C., for 30 (EBI3), 32 (p28), 35 (IL-27R), and 30 (gp130 and β2-microglobulin) cycles.

### IV. Co-Immunoprecipitation and Western Blot Analysis

Frozen placental tissues were grinded and lysed for 1 hr on ice by incubation in lysis buffer (0.5% Nonidet P-40, 50 mM Tris, pH7.4, 150 mM NaCl, 3% glycerol, 1.5 mM EDTA) containing protease inhibitors (1 mM PMSF, 1 μg/ml pepstatin, 10 μg/ml leupeptin). Cell lysate was centrifuged for 15 min at 13,000×g. The supernatant was then pre-cleared with protein A-Sepharose (Amersham Biosciences, Saclay, France) for 2 hours at 4° C. and incubated with rabbit polyclonal anti-EBI3 or anti-p28 antibodies (Abs), or with control rabbit IgG Abs (Sigma, St-Quentin-Fallavier, France) for 2 hours at 4° C. Rabbit polyclonal anti-EBI3 or anti-p28 Abs were purified from the sera of rabbits immunized with a bacterial 6His-EBI3 fusion protein (see, e.g., Devergne et al. (2001) Am J Pathol 159:1763-1776) or with a peptide mapping the N-terminus of p28 (see, e.g., Larousserie et al. (2004) J Pathol 202:164-171), respectively. Immune complexes were collected by incubation with protein A-sepharose for 1 hour at 4° C. Beads were then washed 5 times with 1 ml of 0.5% NP-40 lysis buffer, and bound proteins were recovered by boiling in SDS sample buffer. Eluted proteins were separated by SDS-PAGE and transferred to nitrocellulose membrane for immunoblotting. EBI3 was detected using mouse 2G4H6 mAb (Devergne et al. (2001) supra) and p28 was detected using affinity-purified rabbit anti-p28 Abs recognizing the C-terminus of p28 (Schering-Plough Biopharma, Palo Alto, Calif.). Binding of mouse or rabbit Abs was detected with horseradish peroxidase-conjugated anti-mouse Abs or horseradish peroxidase-conjugated protein A, respectively (Amersham Biosciences). Peroxidase reaction was developed with chemiluminescence reagents (Pierce, Rockford, Il., USA).

### V. ELISA

Culture supernatants from placental explants from 14 normal term placentae, prepared as previously described (Devergne, et al. (2001) supra) and sera from 7 women with normal pregnancy collected at various times during pregnancy (from 9 to 40 weeks of pregnancy), all of which had been previously tested for EBI3 by ELISA (Devergne et al. (2001) supra) were tested for IL-27 by ELISA. IL-27 ELISA, which recognizes EBI3/p28 heterodimer, but not free EBI3, was previously described (see, e.g., Pflanz, et al. (2002) supra).

### VI. Immunohistochemistry

Immunohistochemistry was performed on formalin-fixed paraffin-embedded tissues by an indirect avidin-biotin peroxidase technique as described (Larousserie, et al. (2004) supra; Larousserie, et al. (2005) supra; Larousserie, et al. (2006) supra; and Devergne, et al. (2001) supra). The peroxidase reaction was developed with 3′-diaminobenzidine and sections were counterstained with Harris hematoxylin. EBI3 was detected using 2G4H6 mouse mAb (IgG2a) at 2 μg/ml, in parallel with an isotype-matched control mAb (RPC5, IgG2a, Cappel Durham, N.C., USA). p28 was detected using affinity-purified rabbit polyclonal anti-p28 antibodies (Schering-Plough Biopharma) at 1-2 μg/ml and normal rabbit IgG (Sigma) was used as a negative control. In some cases, rat anti-p28 monoclonal Abs (10 μg/ml) (Schering-Plough Biopharma) were used, in parallel with normal rat IgG, and detected with biotinylated rabbit anti-rat polyclonal IgG (Vector laboratories) as described (Larousserie, et al. (2004) supra).

### VII. Animal Model of Immune Mediated Abortion

The DBAxCBA abortion prone mouse model is the standard model for the study of immune mediated spontaneous abortion (Toder et al., 1989, J Reprod Fertil Suppl 37, 79-84). In pregnant CBA female mice, the resorption rate of DBA:CBA hybrid embryos occur in 21-30% of the embryos compared with 8% resorption in control Balb C: CBA embryos. In this model system, a maternal immunological rejection response towards the embryo initiates at day 6 of gestation, 2 days following implantation (Duclos et al., 1995, Am J Reprod Immunol 33, 354-66). To assess the potential of an IL-27 agonist to suppress maternal rejection of the embryo, IL-27 agonist is administered to pregnant mice with high rates with either high or low rates of spontaneous abortion. Doses are administered at various timepoints, eg., days 4 and 6 of gestation, corresponding to the time of implantation (day 4) and day when immunological reactions to the embryo is first detected (day 6). Quantitation of resorbed embryos is performed on day 12 of gestation. In highly aborting CBA×DBA mated mice (n=14), embryonic resorption rates are compared to those of control Balb/C×DBA mated mice.

All references cited herein are incorporated herein by reference to the same extent as if each individual publication, patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

1. A method of inducing maternal tolerance to a fetal antigen comprising administering to a subject at risk of or suffering from an immune mediated abortion, an effective amount of an IL-27 agonist.

2. The method of claim 1, wherein the subject is human and the IL-27 agonist is administered during:
a) first trimester of pregnancy;
b) second trimester of pregnancy;
c) third trimester of pregnancy; or
d) first, second and third trimesters of pregnancy.

3. The method of claim 1, wherein the IL-27 agonist is IL-27 protein.

4. The method of claim 3, wherein the IL-27 protein is human IL-27 protein.

5. The method of claim 1, wherein the IL-27 agonist is an agonist antibody that binds at least one subunit of an IL-27 receptor (IL-27R) complex.

6. The method of claim 5, wherein the IL-27R complex is a human IL-27R complex.

7. The method of claim 5, wherein the agonist antibody induces signaling of the IL-27R complex.

8. A method of enhancing implantation of at least one embryo to a uterine lining comprising administering to a subject an effective amount of an IL-27 agonist.

9. The method of claim 8, wherein the subject is human and IL-27 agonist is administered during:
a) first trimester of pregnancy;
b) second trimester of pregnancy;
c) third trimester of pregnancy; or
d) first, second, and third trimesters of pregnancy.

10. The method of claim 8, wherein the IL-27 agonist is an IL-27 protein.

11. The method of claim 10, wherein the IL-27 protein is human IL-27 protein.

12. The method of claim 8, wherein the IL-27 agonists is an agonist antibody that binds at least one subunit of an IL-27R complex.

13. The method of claim 12, wherein the IL-27 complex is a human IL-27 complex.

14. The method of claim 12, wherein the agonist antibody induces signaling of the IL-27R complex.

15. A diagnostic method for determining whether a subject is suffering from immune mediated abortion comprising detecting the presence or level of IL-27 or IL-27R in a biological sample obtained from the subject, and determining whether the subject is having a spontaneous abortion.

16. The method of any of claim 15, wherein the biological sample is selected from the group consisting of a tissue sample, a cell sample, or a serum sample.

17. The method of claim 16, wherein the tissue sample is selected from the group consisting of a chorionic villus sample and a placental sample.

18. A prognostic method for determining whether a subject is at risk for developing immune mediated abortion comprising detecting the presence or level of IL-27 or IL-27R mRNA or polypeptide in a biological sample obtained from the subject, or isolate of the sample, thereby determining whether the subject is at risk for developing spontaneous abortion.

19. The method of claim 18, wherein the biological sample is selected from the group consisting of a tissue sample, a cell sample, or a serum sample.

20. The method of claim 19, wherein the tissue sample is selected from the group consisting of a chorionic villus sample and a placental sample.

21. A method of monitoring IL-27 agonist treatment of a subject at risk of or suffering from an immune mediated abortion comprising measuring the levels of a Th1 or Th2 cytokine mRNA or polypeptide in a biological sample from the subject or an isolate of the sample, thereby determining if the subject is at risk of or suffering from an immune mediated abortion.

22. The method of claim 21, wherein the Th1 cytokine is selected from the group consisting of TNF-α, IFN-γ, IL-2, IL-1, IL-6, IL-12, and RANTES.

23. The method of claim 21, wherein the Th2 cytokine is selected from the group consisting of CSF-1, GM-CSF, IL-10, IL-4, IL-11, TGF and IL-3.
